SEQUENCE LISTING (5 sequences)

SEQ ID NO: 1 - protein, 648 aa, unidentified
  source 1..648: note = Parvabulbium thermostercus
MAPAVGIDLGTTYSCVGVFREDRCEIIANDQGNRTTPSFVAFTDTERLIGDAAKNQVAMNPHNTVFDAKRLIGRRFNDPEVQSDMKHFPFKVVDRNGKPAIEVEFKGEKKVFTPEEISSMVLTKMRETAESYLGTTVTNAVITVPAYFNDSQRQATKDAGLIAGLNVLRIINEPTAAAIAYGLDKKADGERNVLIFDLGGGTFDVSLLTIEEGIFEVKSTAGDTHLGGEDFDNRLVNHLCQEFKRKFKKDLTTNARALRRLRTAAERAKRTLSSAAQTSIEIDSLFEGIDFYTSITRARFEELCQDLFRSTLQPVDRVLTDAKIDKSQVHEIVLVGGSTRIPRIQKLITDYFNGKEPNKSINPDEAVAYGAAVQAAILSGDTTSKSTNEILLLDVAPLSLGIETAGGIMTKLIPRNTTIPTKKSEVFSTFSDNQPGVLIQVYEGERQRTKDNNLLGKFELTGIPPAPRGVPQIEVTFDLDANGIMNVSALEKGTGKTNQIVITNDKGRLSKEEIERMLAEAEKFKEEDEREARRVQAKNGLESYAYSLRNTLNDPKVDEKLDASDKEKLRSEIEKIVNWLDENQQASREEYEDQQKELESIANPIMMKFYGAGAGGMPGGMPGGAPGGFPSAGAGSHDNEGPTVEEVD

SEQ ID NO: 2 - protein, 664 aa, synthetic construct
HHHHHHSSGLVPRGSHMAPAVGIDLGTTYSCVGVFREDRCEIIANDQGNRTTPSFVAFTDTERLIGDAAKNQVAMNPHNTVFDAKRLIGRRFNDPEVQSDMKHFPFKVVDRNGKPAIEVEFKGEKKVFTPEEISSMVLTKMRETAESYLGTTVTNAVITVPAYFNDSQRQATKDAGLIAGLNVLRIINEPTAAAIAYGLDKKADGERNVLIFDLGGGTFDVSLLTIEEGIFEVKSTAGDTHLGGEDFDNRLVNHLCQEFKRKFKKDLTTNARALRRLRTAAERAKRTLSSAAQTSIEIDSLFEGIDFYTSITRARFEELCQDLFRSTLQPVDRVLTDAKIDKSQVHEIVLVGGSTRIPRIQKLITDYFNGKEPNKSINPDEAVAYGAAVQAAILSGDTTSKSTNEILLLDVAPLSLGIETAGGIMTKLIPRNTTIPTKKSEVFSTFSDNQPGVLIQVYEGERQRTKDNNLLGKFELTGIPPAPRGVPQIEVTFDLDANGIMNVSALEKGTGKTNQIVITNDKGRLSKEEIERMLAEAEKFKEEDEREARRVQAKNGLESYAYSLRNTLNDPKVDEKLDASDKEKLRSEIEKIVNWLDENQQASREEYEDQQKELESIANPIMMKFYGAGAGGMPGGMPGGAPGGFPSAGAGSHDNEGPTVEEVD

SEQ ID NO: 3 - DNA, 1947 bp, unidentified
  source 1..1947: mol_type = other DNA; note = Parvabulbium thermostercus
atggctcccgccgtcggcattgacttgggcacgacctactcgtgcgtgggcgtgttccgcgaggatcgctgcgaaatcatcgccaatgaccagggcaaccggactactccgtcgttcgtggccttcacggacacggagcgtctgatcggtgatgccgccaagaaccaggtcgccatgaaccctcacaacaccgtcttcgacgccaagcgcctcatcggccgccggttcaacgaccccgaggtccagtccgacatgaagcacttcccgttcaaggtcgttgatcggaacggcaagccggccatcgaggtcgagttcaagggcgagaagaaggtcttcactcccgaggagatctcgtccatggtcctgaccaagatgcgcgagacggccgagtcgtacctgggcaccaccgtcaccaacgccgtcatcaccgtccctgcctacttcaacgactcccaacgccaggccaccaaggacgctggtctgattgccggcctcaacgtcctgcgtatcatcaacgagcccaccgccgctgccattgcctacggtctcgacaagaaggccgatggtgagcgcaacgtcctgatcttcgacctcggtggtggcaccttcgatgtctcgctcctcaccatcgaggaaggcatcttcgaggtcaagtccaccgctggtgacacccacctgggtggtgaggacttcgacaaccggctcgtgaaccatttatgccaagaatttaagcgcaagttcaagaaggacctcacgaccaatgcacgcgccctgcgccgcctgcgcaccgctgccgagcgtgccaagcgcactctatcgagcgctgcccagaccagcatcgagatcgactcgctcttcgagggtattgacttctacacgtcgatcacccgtgctcgtttcgaggagctctgccaggacctgttccgttccaccctccagccggtggaccgcgtcctcacggatgccaagatcgacaagtcgcaggtccatgagatcgtgctcgtcggcggctcgacccgtatcccgcgcatccagaagctcatcaccgactacttcaacggcaaggagcccaacaagtccatcaaccccgacgaggccgttgcctacggcgccgccgttcaggccgccatcctgtcgggcgacacgacttctaagtcgaccaacgagatcctgcttctcgatgtcgctcctctgtcgctcggcatcgagaccgctggcggcatcatgaccaagctcatcccgcgcaacacgaccatcccgaccaagaagtccgaggtgttctcgaccttctccgacaaccagcccggtgtgctcatccaggtctacgagggtgagcgccagcgcaccaaggacaacaacctgctcggcaagttcgagcttaccggcattccgccggctcctcgcggtgtgccccagatcgaggtcacattcgacctggatgccaacggcatcatgaacgtgtcggccctcgagaagggcacgggcaagacgaaccagatcgtcattaccaatgacaagggccgcctgtccaaggaggagattgagcgcatgcttgccgaggccgagaagttcaaggaggaggacgagcgcgaggctcgccgtgtccaggccaagaacggccttgagtcgtacgcctactcgctgcgcaacacgctcaacgaccccaaggtggacgagaagctcgacgcctccgacaaggagaagctgcgctccgagatcgaaaagattgtcaactggctcgacgagaaccagcaggccagccgcgaggagtacgaggaccaacagaaggagctcgagtccattgccaacccgatcatgatgaagttctatggtgctggcgccggtggcatgcccggtggcatgcccggtggcgcccctggtggcttccccagcgccggtgccggcagccacgacaacgagggcccgactgtcgaggaggtcgactaa

SEQ ID NO: 4 - DNA, 1995 bp, synthetic construct
  source 1..1995: mol_type = other DNA
catcatcatcatcatcacagcagcggcctggtgccgcgcggcagccatatggctcccgccgtcggcattgacttgggcacgacctactcgtgcgtgggcgtgttccgcgaggatcgctgcgaaatcatcgccaatgaccagggcaaccggactactccgtcgttcgtggccttcacggacacggagcgtctgatcggtgatgccgccaagaaccaggtcgccatgaaccctcacaacaccgtcttcgacgccaagcgcctcatcggccgccggttcaacgaccccgaggtccagtccgacatgaagcacttcccgttcaaggtcgttgatcggaacggcaagccggccatcgaggtcgagttcaagggcgagaagaaggtcttcactcccgaggagatctcgtccatggtcctgaccaagatgcgcgagacggccgagtcgtacctgggcaccaccgtcaccaacgccgtcatcaccgtccctgcctacttcaacgactcccaacgccaggccaccaaggacgctggtctgattgccggcctcaacgtcctgcgtatcatcaacgagcccaccgccgctgccattgcctacggtctcgacaagaaggccgatggtgagcgcaacgtcctgatcttcgacctcggtggtggcaccttcgatgtctcgctcctcaccatcgaggaaggcatcttcgaggtcaagtccaccgctggtgacacccacctgggtggtgaggacttcgacaaccggctcgtgaaccatttatgccaagaatttaagcgcaagttcaagaaggacctcacgaccaatgcacgcgccctgcgccgcctgcgcaccgctgccgagcgtgccaagcgcactctatcgagcgctgcccagaccagcatcgagatcgactcgctcttcgagggtattgacttctacacgtcgatcacccgtgctcgtttcgaggagctctgccaggacctgttccgttccaccctccagccggtggaccgcgtcctcacggatgccaagatcgacaagtcgcaggtccatgagatcgtgctcgtcggcggctcgacccgtatcccgcgcatccagaagctcatcaccgactacttcaacggcaaggagcccaacaagtccatcaaccccgacgaggccgttgcctacggcgccgccgttcaggccgccatcctgtcgggcgacacgacttctaagtcgaccaacgagatcctgcttctcgatgtcgctcctctgtcgctcggcatcgagaccgctggcggcatcatgaccaagctcatcccgcgcaacacgaccatcccgaccaagaagtccgaggtgttctcgaccttctccgacaaccagcccggtgtgctcatccaggtctacgagggtgagcgccagcgcaccaaggacaacaacctgctcggcaagttcgagcttaccggcattccgccggctcctcgcggtgtgccccagatcgaggtcacattcgacctggatgccaacggcatcatgaacgtgtcggccctcgagaagggcacgggcaagacgaaccagatcgtcattaccaatgacaagggccgcctgtccaaggaggagattgagcgcatgcttgccgaggccgagaagttcaaggaggaggacgagcgcgaggctcgccgtgtccaggccaagaacggccttgagtcgtacgcctactcgctgcgcaacacgctcaacgaccccaaggtggacgagaagctcgacgcctccgacaaggagaagctgcgctccgagatcgaaaagattgtcaactggctcgacgagaaccagcaggccagccgcgaggagtacgaggaccaacagaaggagctcgagtccattgccaacccgatcatgatgaagttctatggtgctggcgccggtggcatgcccggtggcatgcccggtggcgcccctggtggcttccccagcgccggtgccggcagccacgacaacgagggcccgactgtcgaggaggtcgactaa

SEQ ID NO: 5 - protein, 6 aa, synthetic construct
HHHHHH

(19) United States
(12) Patent Application Publication
(10) Pub. No.: US 2025/0304630 A1
(43) Pub. Date: Oct. 2, 2025

(54) POLYPEPTIDES HAVING OSMOPROTECTIVE ACTIVITY, COMPOSITIONS THEREOF AND USES THEREOF

(71) Applicant: DELAVIE SCIENCES, LLC, Worcester, MA (US)

(72) Inventors: Keith BALLARD, Worcester, MA (US); Kyle LANDRY, Worcester, MA (US)

(21) Appl. No.: 18/864,276

(22) PCT Filed: May 11, 2023
(86) PCT No.: PCT/US2023/021881

§ 371 Date: Nov. 8, 2024

Related U.S. Application Data
(60) Provisional application No. 63/341,131, filed on May 12, 2022.

(51) Int. Cl. C07K 14/37; A61K 38/00

(52) U.S. Cl. CPC C07K 14/37; A61K 38/00

(57) ABSTRACT

The technology relates in part to polypeptides having osmoprotective activity and compositions thereof for a variety of uses, including for cosmetic use and for the treatment of disorders, such as inflammatory disorders, which are associated with osmotic stress, or for use as an antioxidant.

### RELATED APPLICATION

This application claims priority to U.S. Provisional Application 63/341,131, filed May 12, 2022, naming Keith Ballard et al. as inventors, titled “Polypeptides Having Osmoprotective Activity, Compositions Thereof and Uses Thereof,” which is incorporated herein by reference in its entirety for all purposes.

### FIELD

The technology relates to polypeptides having osmoprotective activity and compositions thereof for a variety of uses, including for cosmetic use and for the treatment of disorders, such as inflammatory disorders, which are associated with osmotic stress.

### BACKGROUND

Osmotic shock or osmotic stress is a physiologic dysfunction caused by a sudden change in the solute concentration around a cell, which causes a rapid change in the movement of water across its cell membrane. Osmotic stress can adversely impact a number of organs and tissues by causing one or more deleterious effects in the cells, including, altering protein structure and function, altering enzyme activities, dehydration, placing stress on the cytoskeleton and the nucleus, increased production of reactive oxygen species, DNA damage, and premature apoptosis/aging of the cells. Osmotic stress has been linked to inflammatory disorders and to the damage/aging of certain tissues such as the skin, mucous membranes and hair. Agents that have a protective effect against osmotic stress (osmoprotective agents) can minimize or eliminate conditions caused by osmotic stress.

### SUMMARY

Provided herein in certain aspects is a polypeptide (which is referred to interchangeably herein as “HSP” or “HSP70,” by virtue of its homology to certain putative heat shock proteins rather than by virtue of biological activity) containing a consecutive sequence of amino acids that is greater than 95% identical to a corresponding consecutive sequence of amino acids set forth in SEQ ID NO:1. In some embodiments, the sequence of amino acids is 96% or more identical to the sequence of amino acids set forth in SEQ ID NO: 1. and in some embodiments, the polypeptide is a polypeptide of SEQ ID NO. 1. In some embodiments, the polypeptide exhibits an osmoprotective enzyme activity.

Also provided herein is a HSP polypeptide that includes a sequence or a portion of a sequence of amino acids that is more than 95% identical, such as 96%, 97%, 98%, or 99% or more identical, or 100% identical, to a corresponding consecutive sequence of amino acids set forth in SEQ ID NO: 1, wherein the sequence or the portion of the sequence exhibits osmoprotective activity. In some embodiments of the polypeptides provided herein, the polypeptide is an isolated, recombinant, or synthetically produced polypeptide.

In embodiments, the recombinantly produced polypeptide is a polypeptide that includes a sequence or a portion of a sequence of amino acids that is more than 95% identical, such as 96%, 97%, 98%, or 99% or more identical, or 100% identical, to a corresponding consecutive sequence of amino acids set forth in SEQ ID NO: 2. Thus, provided herein in certain aspects is a HSP polypeptide containing a consecutive sequence of amino acids that is greater than 95% identical to a corresponding consecutive sequence of amino acids set forth in SEQ ID NO:2 (also referred to interchangeably herein as HSP or HSP70 for the reason as provided above). In some embodiments, the sequence of amino acids is 96% or more identical to the sequence of amino acids set forth in SEQ ID NO: 2, and in some embodiments, the polypeptide is a polypeptide of SEQ ID NO. 2. Also provided herein is a HSP polypeptide that includes a sequence or a portion of a sequence of amino acids that is more than 95% identical, such as 96%, 97%, 98%, or 99% or more identical, or 100% identical, to a corresponding consecutive sequence of amino acids set forth in SEQ ID NO: 2, wherein the sequence or the portion of the sequence exhibits osmoprotective activity.

Also provided herein in certain aspects are compositions, e.g., polypeptide compositions, that include one or more polypeptides selected from among the polypeptides of SEQ ID NO: 1 or SEQ ID NO:2 and polypeptides having consecutive sequences that are more than 95% identical to a corresponding consecutive sequence of SEQ ID NO:1 or SEQ ID NO:2, or polypeptides that include a sequence or a portion of a sequence of amino acids that is more than 95% identical, such as 96%, 97%, 98%, or 99% or more identical, or 100% identical, to a corresponding consecutive sequence of amino acids set forth in SEQ ID NO: 1 or SEQ ID NO: 2. Included among the compositions provided herein are pharmaceutical compositions, e.g., for the treatment of certain inflammatory diseases or conditions, including conditions associated with inflammation, and cosmetic compositions. e.g., for administration or application to the skin, e.g., as a moisturizer or to minimize DNA damage caused by osmotic stress, or for administration or application to the scalp, such as for reducing hair loss or promoting hair growth in alopecia caused as a side effect of chemotherapy. Also provided herein are combinations of one or more polypeptides provided herein and one or more agents, such as a therapeutically active agent for treating an inflammatory disorder or condition (e.g., a therapeutically active agent that does not include a polypeptide provided herein) and/or a cosmetic agent (e.g., an anti-wrinkle agent). In one aspect, a combination provided herein includes one or more agents, such as, e.g., two, separate agents (for example a polypeptide provided herein and a separate therapeutically active agent or cosmetic agent) for combined treatment or administration. Such agents of a combination can be administered or applied as separate substances but for combined use. For example, such separate agents can be administered serially, sequentially or concurrently but as separate agents that are not mixed prior to administration. In some aspects, the agents of a combination may be mixed together and administered as a single composition.

In certain aspects, a polypeptide, composition or combination provided herein is osmoprotective. In aspects, one or more polypeptides of the compositions or combinations provided herein are osmoprotective. The term “osmoprotective,” as used herein, refers to the composition as a whole, or to one or more polypeptides of the composition, that when administered to a cell under osmotic stress, increases the viability of the cell (lifetime prior to apoptosis) by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%, 185%, 190%, 195%, 200%, 300%, 400% or more, relative to the viability of the cell under osmotic stress that is not subjected to treatment with one or polypeptides, or compositions thereof, as provided herein.

The polypeptides provided herein and/or used in the compositions provided herein can be isolated and/or otherwise obtained from a number of microbes, such as bacteria and fungi, or can be obtained recombinantly or through synthetic production processes. In certain aspects, the polypeptides provided herein and/or used in the compositions provided herein can be obtained from thermophilic microbes, i.e., microbes that can survive at high temperatures greater than or about 30° C., such as 30° C. to 125° C. or more, generally about 30° C. to about 37° C., 38° C., 39° C., 40° C., 41° C. 42° C., 43° C., 44° C. or 45° C. or greater, or about 40° C. to about 120° C. or about 45° C. to about 125° C., or about 37° C. 38° C., 39° C., 40° C., 41° C., 42° C., 43° C., 44° C. or 45° C., and/or contain polypeptides that are stable and/or active at high temperatures greater than or about 30° C., such as 30° C. to 125° C. or more, generally about 30° C. to about 37° C., 38° C., 39° C., 40° C., 41° C., 42° C., 43° C., 44° C. or 45° C. or greater, or about 40° C. to about 120° C. or about 45° C. to about 125° C., or about 37° C., 38° C., 39° C., 40° C., 41° C., 42° C., 43° C., 44° C. or 45° C. For example, the HSP polypeptides provided herein and formulated in the compositions provided herein can be obtained from a thermophilic fungus, TM-417 (see, e.g., Salar, R. K., Thermophilic Fungi: Basic Concepts and Biotechnological Applications, CRC Press (2018), p. 282; submitted to the Index Fungorum as Parvabulbium thermostercus (Index Fungorum, Landry et al. 2021)).

In some aspects, the compositions provided herein, for example, pharmaceutical compositions, can be used to treat a disease or condition in a subject, such as an inflammatory disorder. In some aspects, the compositions provided herein can be administered for cosmetic use. A “subject,” as used herein, can be an animal, such as a human being. As used herein, “treating,” with reference to treating a subject with a disease or condition, including a cosmetic condition or other condition affecting, e.g., the skin, mucous membranes or scalp/hair, means that the disease or condition, or symptoms associated with the disease or condition, are partially or totally alleviated, or the disease or condition and/or symptoms thereof remain static (do not progress) following treatment. Hence, treatment of a disease or condition encompasses prophylaxis, amelioration, reduction or elimination of the disease or condition or symptoms thereof, therapy and/or cure. Prophylaxis refers to prevention of a potential disease and/or a prevention of worsening of symptoms or progression of a disease or condition. As used herein, “treatment” means any manner, e.g., by administration of a composition, e.g., a pharmaceutical composition or cosmetic composition provided herein and, optionally, an additional therapeutically active agent and/or cosmetic treatment agent or other agent, such as an adjuvant, by which a condition, disorder or disease, or symptoms thereof, are prevented, ameliorated, reduced or eliminated.

Thus, in certain aspects, provided herein is a pharmaceutical composition that includes: (a) one or more HSP polypeptides containing the sequence of amino acids set forth in SEQ ID NO:1 or SEQ ID NO:2 and/or containing a consecutive sequence of amino acids that is more than 95% identical to a corresponding consecutive sequence of amino acids set forth in SEQ ID NO:1 or SEQ ID NO:2, and/or selected from among the polypeptides of SEQ ID NO:1 or SEQ ID NO:2 and polypeptides having consecutive sequences that are more than 95% identical to a corresponding consecutive sequence of SEQ ID NO:1 or SEQ ID NO:2, or polypeptides that include a sequence or a portion of a sequence of amino acids that is more than 95% identical, such as 96%, 97%, 98%, or 99% or more identical, or 100% identical, to a corresponding consecutive sequence of amino acids set forth in SEQ ID NO:1 or SEQ ID NO: 2; and (b) a pharmaceutically acceptable excipient, wherein the pharmaceutical composition exhibits osmoprotective activity. Also provided, in some aspects, is a composition for cosmetic use, which includes: (a) one or more HSP polypeptides containing the sequence of amino acids set forth in SEQ ID NO:1 or SEQ ID NO: 2 and/or containing a consecutive sequence of amino acids that is more than 95% identical to a corresponding consecutive sequence of amino acids set forth in SEQ ID NO: 1 or SEQ ID NO:2, and/or selected from among the polypeptides of SEQ ID NO:1 or SEQ ID NO:2 and polypeptides having consecutive sequences that are more than 95% identical to a corresponding consecutive sequence of SEQ ID NO:1 or SEQ ID NO:2, or polypeptides that include a sequence or a portion of a sequence of amino acids that is more than 95% identical, such as 96%, 97%, 98%, or 99% or more identical, or 100% identical, to a corresponding consecutive sequence of amino acids set forth in SEQ ID NO: 1 or SEQ ID NO: 2; and (b) an excipient or adjuvant, wherein the composition exhibits osmoprotective activity.

The compositions provided herein, and combinations provided herein, can contain, in certain aspects, a single polypeptide, or can contain, in aspects, a plurality of polypeptides. In certain aspects, the compositions provided herein, can consist essentially of a single polypeptide selected from among the polypeptides provided herein. In aspects, the single polypeptide is a HSP polypeptide selected from among the polypeptides of SEQ ID NO:1 or SEQ ID NO:2 and polypeptides having consecutive sequences that are more than 95% identical to a corresponding consecutive sequence of SEQ ID NO:1 or SEQ ID NO:2, or polypeptides that include a sequence or a portion of a sequence of amino acids that is more than 95% identical, such as 96%, 97%, 98%, or 99% or more identical, or 100% identical, to a corresponding consecutive sequence of amino acids set forth in SEQ ID NO: 1 or SEQ ID NO: 2. The phrase “consist(s) essentially of,” as used herein, means that components other than the recited components, if present, do not materially alter the activity of the recited components. Thus, for example, in the context of the compositions provided herein, the compositions may include one or more components other than a single polypeptide having the aforementioned sequence characteristics and properties. In some aspects, the compositions, e.g., pharmaceutical compositions or compositions for treating an inflammatory disorder or for cosmetic use or for reducing/protecting from osmotic stress, provided herein consist of a single polypeptide.

The compositions provided herein, such as pharmaceutical compositions or cosmetic compositions as provided herein, and combinations as provided herein, can be in any form, including, but not limited to, a liquid, solid, semi-solid or mixture of a liquid, solid and/or semi-solid. For example, the compositions and combinations can be, or include, a solution, dispersion, suspension, emulsion, or colloid. In aspects, the compositions provided herein, and combinations provided herein, can be formulated in a variety of ways including, but not limited to, a gel, ointment, cream, lotion, oil, butter, paste, balm, stick, foam, serum, mousse, patch, spray, aerosol, powder, or lyophile (or lyophilizate). In aspects, the compositions provided herein, and combinations provided herein, can be formulated as a sustained release formulation. In certain aspects, the formulations provided herein can be formulated for a single administration. In some aspects, the formulations provided herein can be formulated for multiple administrations.

In certain aspects, the compositions provided herein, such as the pharmaceutical compositions or cosmetic compositions provided herein, and combinations provided herein, include an additional agent. For example, the pharmaceutical compositions can be administered along with one or more additional agents for treating inflammatory disorders or conditions characterized by inflammation, or an additional osmoprotective agent or an agent that inhibits or reduces osmotic stress, as a single formulation or as separate formulations. For example, the cosmetic compositions provided herein can be administered with one or more additional cosmetic agents, such as an anti-wrinkle agent.

Provided herein are methods of treating a disease or conditions in a subject, such as a disease or condition characterized by inflammation, or an inflammatory disorder, that includes administering, to a subject in need thereof, one or more polypeptides, isolated or otherwise obtained (e.g., recombinantly or synthetically produced), as provided herein, or one or more pharmaceutical compositions containing the one or more polypeptides provided herein. In some aspects, the method includes administering, to a subject in need thereof, a therapeutically effective amount of one or more of the polypeptides provided herein, and/or one or more of the pharmaceutical compositions provided herein. In certain aspects, the disease or disorder is selected from among inflammatory bowel disease, hypernatremia, dry eye syndrome, tissue burns, dehydration, heat stroke, diabetes mellitus, diabetes insipidus, uremia, kidney disease, cardiovascular disease and liver disease. Also provided herein are methods of improving skin health or appearance, or scalp health or appearance, by administering the polypeptides and/or compositions provided herein as a cosmetic use, such as for administering to the skin, e.g., to counter dehydration, for improving skin tone, for preventing other osmotic stress-induced damage, such as DNA damage, and the like. In some aspects, the methods include administering, to a subject in need thereof, an effective amount of one or more of the polypeptides provided herein, and/or one or more of the cosmetic compositions provided herein. In certain aspects of the methods provided herein, there is osmotic stress associated with the disease or condition or, in the case of cosmetic use, such as for application to the skin, osmotic stress associated with the condition of the skin. In some aspects, the osmotic stress is reduced by at least about 10%, 15%, 20%, 25%, 30%, 35%, 40%, 45%, 50%, 55%, 60%, 65%, 70%, 75%, 80%, 85%, 90%, 95%, 100%. 125%, 150%, 155%, 160%, 165%, 170%, 175%, 180%, 185%, 190%, 195%, 200%, 300%, 400% or more, as measured, for example, in an osmotic stress assay, by an increase in viability of a cell exposed to hypertonic conditions by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%, 185%, 190%, 195%, 200%, 300%, 400% or more, upon treatment using the methods provided herein.

Also provided herein are methods of reducing osmotic stress in a cell by administering an effective amount of one or more of the polypeptides provided herein, and/or one or more of the compositions provided herein. In some aspects, the osmotic stress is reduced by by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%, 185%, 190%, 195%, 200%, 300%, 400% or more, as measured, for example, in an osmotic stress assay, by an increase in viability of a cell exposed to hypertonic conditions by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%. 185%, 190%, 195%, 200%, 300%, 400% or more. The cell can be in a culture, i.e., in vitro, or the cell can be in an animal or human subject.

Also provided herein are methods of treating a disease or conditions in a subject, such as a disease or condition characterized by inflammation, or an inflammatory disorder, that includes administering, to a subject in need thereof, one or more polypeptides, isolated or otherwise obtained (e.g., recombinantly or synthetically produced), as provided herein, or one or more pharmaceutical compositions containing the one or more polypeptides provided herein, and one or more additional active agents for treating the condition or disorder. In some aspects, the method includes administering, to a subject in need thereof, a therapeutically effective amount of one or more of the polypeptides provided herein, and/or one or more of the pharmaceutical compositions provided herein, and/or one of more of the additional tactive agents. In some aspects, the additional active agent is an agent that reduces osmotic stress, or is osmoprotective. In aspects, the agent that reduces osmotic stress, or is osmoprotective includes, but is not limited to, sorbitol, myoinositol, a heat shock protein other than the HSP polypeptides provided herein, or an inhibitor of aldose reductase (AR), e.g., fidarestat. In certain aspects, the disease or disorder is selected from among inflammatory bowel disease, hypernatremia, dry eye syndrome, tissue burns, dehydration, heat stroke, diabetes mellitus, diabetes insipidus, uremia, kidney disease, cardiovascular disease and liver disease. Also provided herein are polypeptides and/or compositions thereof for cosmetic use, such as for administering to the skin, e.g., to counter dehydration, for improving skin tone, skin disorders such as epidermolysis bulosa that cause blistering of the skin, for preventing other osmotic stress-induced damage to the skin, such as DNA damage, and the like, or for administration or application to the scalp, such as for reducing hair loss or promoting hair growth in alopecia caused as a side effect of chemotherapy, and an additional agent, e.g., an anti-wrinkle agent for the skin, or an additional agent to prevent hair loss or facilitate hair growth (e.g., in alopecia). In some aspects, the methods include administering, to a subject in need thereof, an effective amount of one or more of the polypeptides provided herein, and/or one or more of the cosmetic compositions provided herein. In certain aspects of the methods provided herein, there is osmotic stress associated with the disease or condition or, in the case of cosmetic use, such as for application to the skin, osmotic stress associated with the condition of the skin. In some aspects, the osmotic stress is reduced by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%, 185%, 190%, 195%, 200%, 300%, 400% or more, as measured, for example, in an osmotic stress assay, by an increase in viability of a cell exposed to hypertonic conditions by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%, 185%, 190%, 195%, 200%, 300%, 400% or more, upon treatment using the methods provided herein.

Also provided herein are devices that include a polypeptide or a composition provided herein, such as a polypeptide or composition for treating a disorder or condition, such as an inflammatory disease or other condition associated with inflammation, or a polypeptide or composition for cosmetic use. In certain aspects, the device can be a wound dressing, a topical patch, a syringe, an inhaler, a dosage cup, a dropper, a pump, a spray bottle, an aerosol container or an applicator for administering the polypeptide or composition.

In certain aspects, provided herein are kits that include a polypeptide or a composition provided herein, and a device for administration of the composition. In some aspects, the polypeptide or composition is contained in the device for administration. In certain aspects, the polypeptide or composition is present as a separate component that is distinct from the device.

In certain aspects, the device included in the kits provided herein can be selected from among a dressing, a topical patch, a pump, a spray bottle, an aerosol container, a syringe, an inhaler, a dosage cup, a dropper, or an applicator.

Also provided herein are polynucleotides encoding a polypeptide that includes the sequence of amino acids set forth in SEQ ID NO:1 or SEQ ID NO:2 and/or includes a consecutive sequence of amino acids that is more than 95% identical, such as 96%, 97%, 98%, or 99% or more identical, or 100% identical, to a corresponding consecutive sequence of amino acids set forth in SEQ ID NO:1 or SEQ ID NO:2, wherein the polynucleotide is: (i) a polynucleotide of SEQ ID NO:3 or a polynucleotide of SEQ ID NO:4, or (ii) a polynucleotide that is more than 90% identical, such as 91%, 92%, 93%, 94%, 95%, 96%, 97%, 98%, or 99% or more identical, or 100% identical to the sequence of nucleotides set forth in SEQ ID NO:3 or SEQ ID NO:4. In certain aspects, a polypeptide encoded by the polynucleotides provided herein exhibits osmoprotective activity.

Also provided herein are vectors containing the polynucleotides provided herein. In certain aspects, a vector provided herein is an expression vector. In aspects, the vector includes the sequence of nucleotides set forth in SEQ ID NO:3 or SEQ ID NO:4, or a polynucleotide that is more than 90% identical, such as 91%, 92%, 93%, 94%, 95%, 96%, 97%, 98%, or 99% or more identical, or 100% identical to the sequence of nucleotides set forth in SEQ ID NO: 3 or SEQ ID NO:4.

### DETAILED DESCRIPTION

### Osmotic Stress

Osmotic shock or osmotic stress is physiologic dysfunction caused by a sudden change in the solute concentration around a cell, which causes a rapid change in the movement of water across its cell membrane. Under hypertonic conditions-conditions of high concentrations of either salts, substrates or any solute in the supernatant-water is drawn out of the cells through osmosis. This also inhibits the transport of substrates and cofactors into the cell thus “shocking” the cell. Alternatively, under hypotonic conditions-when concentrations of solutes are low-water enters the cell in large amounts, causing it to swell and either burst or undergo apoptosis.

The ability to regulate and preserve distinct intracellular and extracellular solute microenvironments is important in maintaining cellular homeostasis. The osmolarity of human serum is restricted within a tightly regulated range (285-295 mOsm/kg) and, by convention, termed isotonic because this describes the extra- and intracellular osmolarity found within most tissues. Fluids with osmolarities above or below this range are referred to as being hypertonic or hypotonic, respectively. Some tissues, including the kidney and gastrointestinal tract, are exposed to significant fluctuations in osmolarity as a direct consequence of normal, physiological processes. An imbalance between extracellular and intracellular fluid osmolarity, and therefore osmotic pressure, is the underlying cause of osmotic stress. When extracellular fluid osmolarity is greater than that of the intracellular fluid, cells and tissues experience hyperosmotic stress. Conversely, hypoosmotic stress describes the situation where intracellular solute concentrations exceed those outside the cell. Such osmotic imbalances detrimentally affect water flux, cell volume, and cell homeostasis. When osmoadaptive responses fail to compensate for solute concentration asymmetry, negative consequences manifest and potentially contribute to inflammation and disease. The issue of hypertonicity and hyperosmotic stress response is well investigated within the kidney owing to the unique environment encountered within this specialized organ. More recently, studies have revealed that non-renal tissues also commonly experience hyperosmotic stress, especially under pathological conditions. Furthermore, a number of studies have found a strong association between microenvironmental hypertonicity and inflammation. In addition to hyperosmotic stress-related pathologies, evidence suggests hypoosmotic stress also can act as an inflammatory stimulus and can be associated with a number of disorders, including acetaminophen toxicity and brain edema (see, e.g., Brocker et al., Biomol. Concepts, 3 (4): 345-364 (2012), and documents cited therein).

An increase in extracellular osmolarity has many damaging effects on cells by promoting water flux out of the cell, triggering cell shrinkage, and intracellular dehydration. The loss of intracellular water adversely affects protein structure and function, a consequence of which is altered enzyme activity. Cell shrinkage places a great deal of mechanical stress on the cytoskeleton as well as on the nucleus. DNA strand breaks trigger activation of growth arrest and DNA damage (GADD)-inducible genes, such as GADD45 and GADD153. p53 expression and activation also increases during exposure to hyperosmotic stress. Together, up-regulation of these proteins results in cell cycle arrest. Protein translation and degradation are also significantly hindered, in addition to transcription. As damage accumulates, cells become primed for, and ultimately undergo, apoptosis. Generally speaking, the degree of damage is proportional to the degree of osmotic imbalance. However, studies have shown that certain compounds exhibit solute-specific effects and, similarly, elicit a solute-specific response. The responsiveness to hyperosmotic stress varies between cell types and tissues, suggesting that, although there may be a general response mechanism shared by cells, pathway activation and overall outcome differs from cell to cell.

Elevated fluid osmolarity can negatively affect cells in a variety of ways. Osmotic imbalance initially manifests as cell shrinkage as water moves out of the cell. Intracellular water loss disrupts many homeostatic processes, including DNA synthesis and repair, transcription, protein translation and degradation, as well as mitochondrial function. As a result, cell cycle progression and cell proliferation are halted. There is a concomitant increase in oxidative stress and activation of apoptotic pathways. Nuclear shrinkage accompanies overall cell shrinkage and the nucleus assumes a convoluted shape. As cell and nuclear volumes decrease, intracellular macromolecule concentrations increase significantly. Nuclear alterations brought about by extracellular changes in osmolarity can have profound effects on many processes, including chromatin condensation and nucleocytoplasmic transport. Hypertonicity causes DNA strand breaks and activates G₂ and G₁ cell cycle check points. Mitogen-activated protein kinase 14 (MAPK14, also termed p38 MAPK) mediates G₂ phase delays in response to increasing NaCl concentrations. The cell cycle delays associated with G₁, as well as S phase, are attributed to ataxia telangiectasia mutated (ATM)-mediated p53 phosphorylation, p21 induction, and retinoblastoma protein hypophosphorylation. The underlying signaling pathways closely parallel those activated during ultraviolet radiation damage.

Hyperosmotic stress and apoptotic cell death are both characterized by cell shrinkage, and there are similarities between the signaling pathways found within the two processes. Increases in hypertonicity are known to trigger both autophagy and apoptosis in vitro and in vivo. Hypertonicity-induced cell death is characterized by many classic apoptotic features, including nuclear condensation, DNA fragmentation, caspase activation, the appearance of apoptotic bodies, and extracellular phosphatidylserine exposure. Both intrinsic and extrinsic apoptotic signaling pathways appear to be activated during prolonged hyperosmotic stress.

Hyperosmotic stress potentially can contribute to a number of human diseases. Many tissues, including kidney, liver, heart, skin and the like, often experience hyperosmotic stress that can contribute significantly to disease initiation and progression, e.g., by triggering inflammation. Osmotic stress (hyperosmotic stress) can cause aging/apopotosis of skin cells and increase susceptibility of the skin to certain types of damage, such as DNA damage, by interfering with the DNA damage response. Moreover, a growing body of evidence implicates hyperosmotic stress as a potent inflammatory stimulus by triggering proinflammatory cytokine release and inflammation. Under physiological conditions, the urine concentrating mechanism within the inner medullary region of the mammalian kidney exposes cells to high extracellular osmolarity. As such, renal cells have developed many adaptive strategies to compensate for increased osmolarity. Hyperosmotic stress is linked to many maladies, including acute and chronic, as well as local and systemic, inflammatory disorders. Hyperosmolarity triggers cell shrinkage, oxidative stress, protein carbonylation, mitochondrial depolarization, DNA damage, and cell cycle arrest, thus rendering cells susceptible to apoptosis.

The polypeptides and compositions provided herein have an osmoprotective effect and can be used to prevent and/or reduce osmotic stress, e.g., in diseases or conditions characterized by inflammation including, but not limited to, inflammatory bowel disease, hypernatremia, dry eye syndrome, tissue burns, dehydration, heat stroke, diabetes mellitus, diabetes insipidus, uremia, kidney disease, cardiovascular disease and liver disease, in skin conditions, e.g., for cosmetic use or for improving skin appearance or health such as preventing/treating dehydration or slowing down aging/apoptosis, and/or for reducing hair loss or promoting hair growth in alopecia caused as a side effect of chemotherapy

### Polypeptides

Provided herein are polypeptides that include the polypeptide of SEQ ID NO:1 or of SEQ ID NO: 2 (referred to interchangeably herein as HSP or HSP70 polypeptides for reasons as provided herein), and polypeptides that include a consecutive sequence of amino acids that is more than 95% identical to a corresponding consecutive sequence of amino acids of SEQ ID NO:1 or SEQ ID NO:2. For example, a consecutive sequence of amino acids of the polypeptides in the compositions provided herein can have at least 96%, 97%, 98% or 99% or more amino acid sequence identity to a corresponding consecutive sequence of amino acids of SEQ ID NO:1 or SEQ ID NO:2. In some aspects, a polypeptide is an isolated and/or purified polypeptide, a recombinant polypeptide or a synthetically produced polypeptide. In certain aspects, the polypeptides provided herein have an osmoprotective effect, such as preventing or reducing osmotic stress, e.g., prevent hyperosmosis or treat hyperosmosis that causes osmotic stress.

The polypeptides can be isolated or otherwise obtained by methods known to those of skill in the art and/or provided herein. For example, the polypeptides can be generated recombinantly or produced using synthetic processes, such as chemical peptide synthesis methods. In addition, for the polypeptides that have a consecutive sequence of amino acids that have less than 100% sequence identity to a corresponding consecutive sequence of amino acids of SEQ ID NO:1 or SEQ ID NO:2, the modified polypeptides and encoding nucleic acid molecules can include conservative or radical (non-conservative) amino acid substitutions, insertions or deletions. In certain aspects, the HSP polypeptides and modified polypeptides can be produced by standard recombinant DNA techniques known to one of skill in the art and described elsewhere herein. Such modified polypeptides for use in the methods and compositions provided herein also can include modifications of the HSP polypeptides provided herein in a manner that improves stability or half-life, e.g., by chemical modification or a post-translational modification, glycosylation, carboxylation, hydroxylation, sulfation, phosphorylation, albumination, farnesylation, multimerization, conjugation to another protein or polypeptide, such as an antibody or antigen-binding fragment thereof, or conjugation to a polymer such as dextran, a polyethylene glycol (pegylation (PEG)) or sialyl moiety, or other such polymers, such as natural or sugar polymers, and other protein modifications known to those of skill in the art. In addition, one or more amino acids (e.g., a consecutive sequence of more than one amino acid) can be included in the polypeptides provided herein to facilitate handling (e.g., isolation, purification) and/or analysis of the polypeptide. Such amino acid(s) additions include tags or other moieties, for example, to aid in detection or affinity purification of the polypeptide. Examples include, but are not limited to, amino acid sequences that can serve as an epitope tag or other detectable marker, or a thrombin cleavage site. Particular non-limiting examples of such sequences include a His tag e.g., 6×His, or a Flag Tag.

The polypeptides contained in the compositions provided herein can be isolated from a microbial organism, such as, for example, a bacterium or fungus. The bacterium or fungus can be a thermophilic microorganism. For example, the HSP polypeptide of SEQ ID NO:1 can be isolated from the thermophilic fungus, TM-417. Thus, in certain aspects, the compositions provided herein and/or one or more polypeptide components of the compositions provided herein are thermally stable.

Methods for production and purification of proteins such as the HSP polypeptides provided herein for use in the compositions, combinations and methods provided herein are known in the art (see, e.g., Tan et al., Biomed. Res. Int., Article ID 574398 (2009)). In addition, for polypeptide components of the compositions provided herein that have a consecutive sequence of amino acids that bears more than 95% sequence identity to a corresponding consecutive sequence of amino acids of the HSP polypeptides of SEQ ID NO:1 or SEQ ID NO: 2, the modified polypeptides and encoding nucleic acid molecules can be produced by standard recombinant DNA techniques known to one of skill in the art. Any method known in the art to effect mutation of any one or more amino acids in a target protein, e.g., an HSP polypeptide, can be employed. Methods can include standard site-directed or random mutagenesis of encoding nucleic acid molecules, or solid phase polypeptide synthesis methods. For example, nucleic acid molecules encoding a HSP polypeptide can be subjected to mutagenesis, such as random mutagenesis of the encoding nucleic acid, error-prone PCR, site-directed mutagenesis, overlap PCR, gene shuffling, or other recombinant methods. The nucleic acids encoding the polypeptides can then be introduced into a host cell to be expressed heterologously. Hence, also provided herein are nucleic acid molecules encoding any of the polypeptides of the compositions provided herein. In some aspects, the polypeptides of the compositions provided herein can be produced synthetically, such as by using solid phase or solution phase peptide synthesis.

Polypeptides such as an HSP polypeptide provided herein also can be obtained by methods well known in the art for recombinant protein expression and purification. An example of a method for recombinant expression and purification of an HSP polypeptide is provided in Example 1. Any method known to those of skill in the art for identification of nucleic acids that encode desired genes can be used. Any method available in the art can be used to obtain a full length (i.e., encompassing the entire coding region) cDNA or genomic DNA clone encoding a polypeptide provided herein, such as from a cell or tissue source. Modified HSP polypeptides, such as those bearing more than 95% consecutive amino acid sequence identity with a corresponding consecutive amino acid sequence in SEQ ID NO:1 or SEQ ID NO:2, can be engineered from a wildtype polypeptide, such as by site-directed mutagenesis.

The polypeptides provided herein can be used in methods, e.g., for the treatment of a disease or condition susceptible to, associated with and/or triggered by inflammation, e.g., an inflammatory disorder, or as a cosmetic composition for the skin or to treat skin conditions, or to reduce hair loss or promote hair growth, e.g., in alopecia caused by chemotherapy. The polypeptides provided herein also can be used in or formulated as a composition provided herein, such as a pharmaceutical composition, e.g., for treating inflammatory disorders, or as a cosmetic composition for the skin or for discouraging hair loss or promoting hair growth, or as a combination in a single composition/formulation or as separate compositions/formulations with an additional agent for treating, e.g., an inflammatory disease or a skin condition, for use in these methods.

### Polynucleotides

The polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a desired polypeptide, such as an HSP polypeptide, including for example, polymerase chain reaction (PCR) methods. A nucleic acid containing material can be used as a starting material from which a desired polypeptide-encoding nucleic acid molecule can be isolated. For example, DNA and mRNA preparations, or crude microbial extracts, such as a TM-417 fungal extract, can be used in amplification methods. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. Nucleic acid molecules generated by amplification can be sequenced and confirmed to encode a desired polypeptide. A polynucleotide encoding an HSP polypeptide obtained by amplification of nucleic acids from TM-417 fungal cells is provided herein as SEQ ID NO:3, and a polynucleotide encoding a recombinantly expressed HSP is provided herein as SEQ ID NO: 4 (includes a His-tag and a thrombin cleavage site for purification purposes).

A polynucleotide encoding a polypeptide provided herein, e.g., an HSP polypeptide of SEQ ID NO:1 or SEQ ID NO:2, can also be synthetically produced using methods known in the art, such as, for example, synthetic chemistry based polynucleotide synthesis methods. Additionally, the polynucleotides encoding a polypeptide provided herein can be modified for any reason, including, for example, to facilitate or enhance expression in a recombinant host. For example, a polynucleotide sequence encoding a polypeptide provided herein can be modified to optimize the codons encoding the amino sequence of the desired polypeptide without altering the amino sequence. Alternatively, a prokaryotic host cell that is designed for compatibility in using eukaryotic codons instead of prokaryotic codons may be used. For example, commerically available Rosetta™ host strains are BL21 derivatives designed to enhance the expression of eukaryotic proteins that contain codons rarely used in E. Coli. These strains supply tRNAs for AGG, AGA, AUA, CUA, CCC, GGA codons on a compatible chloramphenicol-resistant plasmid. In this way, the Rosetta™ strains provide for “universal” translation which is otherwise limited by the codon usage of E. Coli. The tRNA genes are driven by their native promoters. In Rosetta™ (DE3)pLysS, the rare tRNA genes are present on the same plasmid that carries the T7 lysozyme gene. In various embodiments, the plasmid expression vector is prepared with a cDNA nucleotide sequence derived from an experimentally obtained amino acid sequence. In various embodiments, the nucleotide sequence reflects codons that are more common in eukaryotic cells. In various embodiments, the nucleotide sequence of a polynucleotide encoding a HSP polypeptide is codon optimized for insertion in plasmid DNA. In various embodiments, the nucleotide sequence is provided herein in SEQ ID NO:4.

Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. Additional nucleotide residues sequences such as sequences of bases specifying protein binding regions also can be linked to enzyme-encoding nucleic acid molecules. Such regions include, but are not limited to, sequences of residues that facilitate or encode proteins that facilitate uptake of an enzyme into specific target cells, or otherwise alter pharmacokinetics of a product of a synthetic gene.

In addition, tags or other moieties can be added, for example, to aid in detection or affinity purification of the expressed polypeptide, such as the HSP polypeptides provided herein. For example, additional nucleotide residues sequences such as sequences of bases specifying an epitope tag or other detectable marker also can be linked to enzyme-encoding nucleic acid molecules. Non-limiting examples of such sequences include nucleic acid sequences encoding a His tag e.g., 6×His, or a Flag Tag.

Also provided herein are polynucleotides encoding a polypeptide that includes the sequence of amino acids set forth in SEQ ID NO:1 or SEQ ID NO:2 and/or includes a consecutive sequence of amino acids that is more than 95% identical to a corresponding consecutive sequence of amino acids set forth in SEQ ID NO:1 or SEQ ID NO:2. In aspects, the polynucleotide is: (i) a polynucleotide of SEQ ID NO:3, (ii) a polynucleotide of SEQ ID NO:4, or (iii) a polynucleotide that encodes a consecutive sequence of amino acids that is more than 95% identical to a consecutive sequence of amino acids set forth in SEQ ID NO:1 or SEQ ID NO:2, such as 96%, 97%, 98% or 99% or more amino acid sequence identity to a corresponding consecutive sequence of amino acids of SEQ ID NO: 1 or SEQ ID NO:2. In certain aspects, the polypeptide encoded by the polynucleotides provided herein exhibits osmoprotective activity, i.e., the ability to prevent, reduce or eliminate osmotic or hyperosmotic stress. In some aspects, such a polynucleotide is at least 90%, 91%, 92%, 93%, 94%, 95%, 96%, 97%, 98%, 99% or 100% identical to a corresponding consecutive sequence of nucleotides set forth in SEQ ID NO:3 or SEQ ID NO: 4.

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pCMV4, pBR322 or pUC plasmid derivatives or the Bluescript vector (Stratagene, La Jolla, CA). Other expression vectors include the HZ24 expression vector. The insertion into a cloning vector can, for example, be accomplished by ligating the DNA fragment into a cloning vector which has complementary cohesive termini. Insertion can be effected using TOPO cloning vectors (Invitrogen, Carlsbad, CA). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and protein gene can be modified by homopolymeric tailing. Recombinant molecules can be introduced into host cells via, for example, transformation, transfection, infection, electroporation and sonoporation, so that many copies of the gene sequence are generated.

For recombinant expression of a protein such as an HSP polypeptide provided herein, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals also can be supplied by the native promoter for enzyme genes, and/or their flanking regions. Also provided are vectors that contain a nucleic acid encoding an HSP polypeptide of SEQ ID NO:1 or SEQ ID NO: 2 and variants that bear more than 95% sequence identity thereto, such as 96%, 97%, 98% or 99% or more amino acid sequence identity to a corresponding consecutive sequence of amino acids of SEQ ID NO: 1 or SEQ ID NO:2. The vectors can be selected for expression of the polypeptide in the cell or such that the polypeptide is expressed as a secreted polypeptide. Cells containing the vectors also are provided. The cells include eukaryotic and prokaryotic cells, and the vectors are any suitable for use therein.

Prokaryotic and eukaryotic cells containing the vectors are provided. Such cells include bacterial cells, yeast cells, fungal cells, Archea, plant cells, insect cells and animal cells.

The cells are used to produce a protein thereof by growing the above-described cells under conditions whereby the encoded polypeptide is expressed by the cell, and recovering the expressed polypeptide.

A variety of host-vector systems can be used to express the coding sequences of the polypeptides of the compositions provided herein. These include, but are not limited to, mammalian cell systems; insect cell systems; microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding protein, or domains, derivatives, fragments or homologs thereof, can be regulated by a second nucleic acid sequence so that the genes or fragments thereof are expressed in a host transformed with the recombinant DNA molecule(s). For example, expression of the polypeptides can be controlled by any promoter/enhancer known in the art. Promoters that can be used include, but are not limited to, the SV40 early promoter, the promoter contained in the 3′ long terminal repeat of Rous sarcoma virus, the herpes thymidine kinase promoter, the regulatory sequences of the metallothionein gene, prokaryotic expression vectors such as the β-lactamase promoter or the tac promoter; see also “Useful Proteins from Recombinant Bacteria”: in Scientific American 242:79-94 (1980)); plant expression vectors containing the nopaline synthetase promoter or the cauliflower mosaic virus 35S RNA promoter, and the promoter of the photosynthetic enzyme ribulose bisphosphate carboxylase; promoter elements from yeast and other fungi such as the Gal4 promoter, the alcohol dehydrogenase promoter, the phosphoglycerol kinase promoter and the alkaline phosphatase promoter.

### Compositions and Combinations

Certain aspects of the compositions and combinations provided herein are now described. In some aspects, provided are compositions and combinations for treating a disease or condition, including those characterized by or associated with inflammation. In certain aspects, the disease or disorder is selected from among inflammatory bowel disease, hypernatremia, dry eye syndrome, tissue burns, dehydration, heat stroke, diabetes mellitus, diabetes insipidus, uremia, kidney disease, cardiovascular disease and liver disease. Also provided are compositions and combinations, including cosmetic compositions and combinations, e.g., for treatment and/or care of the skin, mucous membranes and/or hair. In certain aspects, the compositions and combinations provided herein have an osmoprotective effect, e.g., prevent hyperosmosis or treat hyperosmosis that causes osmotic stress.

The compositions and combinations provided herein can include one or more HSP polypeptides selected from among the polypeptides of SEQ ID NO:1 and SEQ ID NO:2, or polypeptides that are more than 95% identical to a corresponding consecutive sequence of amino acids of SEQ ID NO:1 or SEQ ID NO:2. For example, a consecutive sequence of amino acids of the polypeptides in the compositions provided herein can have at least 96%, 97%, 98% or 99% or more amino acid sequence identity to a corresponding consecutive sequence of amino acids of SEQ ID NO:1 or SEQ ID NO:2. In certain aspects, the polypeptide is an isolated and/or purified, recombinant or synthetically produced polypeptide. A recombinant polypeptide is one that is produced using genetic recombination/engineering technologies and can involve heterologous expression of a polynucleotide encoding the polypeptide in a host cell system, e.g., heterologous host cell. A synthetically produced polypeptide is one that is generated using synthetic chemistry methods. The compositions and combinations provided herein can be pharmaceutical compositions or combinations, e.g., for the treatment of diseases or conditions that are caused by or associated with inflammation, or they can be compositions or combinations for cosmetic or therapeutic use, e.g., for treatment and/or care of the skin, mucous membranes and/or hair.

The compositions and combinations provided herein and/or one or more polypeptide components of the compositions and combinations provided herein can, in certain aspects, have osmoprotective activity, i.e., they prevent or reduce osmotic stress in a cell.

The polypeptides, compositions and combinations provided herein can be formulated for direct administration, or application, or can require dilution. They can be formulated for multiple or single dosage (or application) administration. Non-limiting examples of compositions include concentrations of polypeptide(s), e.g., HSP and/or and variants having a consecutive sequence of amino acids that bears more than 95% sequence identity to a HSP polypeptide of SEQ ID NO:1 or SEQ ID NO:2, such as 96%, 97%, 98%, or 99% or more identical, or 100% identity to a corresponding consecutive sequence of amino acids set forth in SEQ ID NO: 1 or SEQ ID NO:2, of between about 0.1 μg/mL to 400 μg/mL, 1 μg/mL to 350 μg/mL, 5 g/mL to 350 μg/mL, 5 μg/mL to 250 μg/mL, 5 μg/mL to 100 μg/mL, 10 μg/mL to 50 μg/mL, 100 μg/mL to 350 μg/mL, 150 μg/mL to 300 μg/mL or 5 μg/mL to 40 μg/mL. Non-limiting examples of compositions also include concentrations of polypeptide(s) provided herein, e.g., HSP and/or variants having a consecutive sequence of amino acids that bears more than 95% sequence identity to a HSP polypeptide of SEQ ID NO:1 or SEQ ID NO:2, such as 96%, 97%, 98%, or 99% or more identical, or 100% identity to a corresponding consecutive sequence of amino acids set forth in SEQ ID NO: 1 or SEQ ID NO:2, of about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, or about 0.1% to about 1% weight/volume of the one or more polypeptides. Further non-limiting examples of compositions also include concentrations of polypeptide(s) provided herein, e.g., the HSP polypeptides and/or variants thereof having a consecutive sequence of amino acids that bears more than 95%, such as 96%, 97%, 98%, or 99% or more identical, or 100% identity to a corresponding consecutive sequence of amino acids set forth in SEQ ID NO: 1 or SEQ ID NO:2, of about 10% or less, 9% or less, 8% or less, 7% or less, 6% or less, 5% or less, 4% or less, 3% or less, 2% or less, 1% or less, 0.5% or less, or 0.25% or less weight/volume of the one or more polypeptides, or about 10%, 9%, 8%, 7%, 6%, 5%, 4%, 3%, 2%, 1%, 0.5% or 0.25% weight/volume of the one or more polypeptides.

In certain aspects, the polypeptides, compositions and combinations provided herein have osmoprotective activity and the concentrations of polypeptide(s) provided herein, including in the compositions and combinations provided herein, are used in an amount that increases the viability of a cel subjected to osmotic stress (lifetime prior to apoptosis) by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%, 185%, 190%, 195%, 200%, 300%, 400% or more, relative to the viability of the cell under osmotic stress that is not subjected to treatment with one or polypeptides, or compositions thereof, as provided herein. An example of an in vitro assay for measuring osmoprotective activity is demonstrated in Example 2.

In some aspects, the compositions can take the form of solutions, suspensions, emulsions, tablets, pills, capsules, powders, and sustained release formulations. Oral formulations, e.g., for pharmaceutical compositions for the treatment of inflammatory diseases and other disorders associated with osmotic stress, can include standard carriers such as pharmaceutical grades of mannitol, lactose, starch, magnesium stearate, sodium saccharine, cellulose, magnesium carbonate, and other such agents. Topical, oral, transdermal and parenteral formulations of any of the compositions provided herein also are contemplated.

Any of the compositions and combinations provided herein can be administered using a device. In certain aspects, the device can be a wound dressing, a topical patch, a syringe, an inhaler, a dosage cup, a dropper, a pump, a spray bottle, an aerosol container or an applicator for administering the composition. In some aspects, the device is a pump for irrigation of a wound or sore with the composition, e.g., pharmaceutical composition, or combination. In aspects, the device is a spray bottle or aerosol container for coating a wound or sore with the composition, or combination. Also provided herein are kits that include compositions or combinations provided herein and a device for administration of the compositions or combinations.

The HSP polypeptides provided herein can be incorporated into the compositions by aqueous solution, and those which are not soluble in water or less soluble in water can be solubilized in cosmetically or pharmaceutically acceptable conventional solvents such as, but not limited to, ethanol, propanol, isopropanol, propylene glycol, glycerine, butylene glycol or polyethylene glycol or any combination thereof. The cosmetically or pharmaceutically effective amount of the HSP polypeptides to be administered, as well as their dosage, can depend on numerous factors, including age, state of the patient, the nature or severity of the condition, disorder or disease to be treated, cared for and/or prevented, the route and frequency of administration and of the particular nature of the peptides to be used.

The terms “cosmetically” or “pharmaceutically” effective amount, as used herein, are understood to mean a nontoxic but sufficient amount of the HSP polypeptides provided herein that will produce the desired cosmetic or pharmaceutical effect. The HSP polypeptides provided herein are provided in cosmetically or pharmaceutically effective concentrations to achieve the desired effect, e.g., between 0.00000001% and 20% of the total weight of the composition, or between 0.000001% and 20% of the total weight of the composition, between 0.0001% and 10% of the total weight of the composition, or between 0.0001% and 5% of the total weight of the composition.

The HSP polypeptides provided herein can also be incorporated into cosmetic or pharmaceutical delivery systems and/or sustained release systems. Delivery systems can include a diluent, adjuvant, excipient or carrier with which the HSP polypeptide(s) are administered. These cosmetic or pharmaceutical carriers can be liquids, such as water, oils or surfactants, including those of petroleum, animal, vegetable or synthetic origin, including but not limited to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, digitonin and the like. Other examples of delivery or sustained release systems include, but are not limited to, liposomes, mixed liposomes, oleosomes, niosomes, ethosomes, milliparticles, microparticles, nanoparticles and solid lipid nanoparticles, nanostructured lipid carriers, sponges, cyclodextrins, vesicles, micelles, mixed micelles of surfactants, surfactant-phospholipid mixed micelles, millispheres, microspheres and nanospheres, lipospheres, millicapsules, microcapsules and nanocapsules, microemulsions, including water-in-oil emulsions with reverse micelle structures, and nanoemulsions, which can be added to achieve a greater penetration of the active ingredient and/or improve its pharmacokinetic and pharmacodynamic properties.

Sustained release systems can be prepared by methods known in the art, and the compositions which contain them can be administered, for example, by topical or transdermal administration, including adhesive patches, non-adhesive patches, occlusive patches and microelectric patches, or by systemic administration, for example and not limited to, orally or parenterally, including nasal, rectal or subcutaneous implantation or injection, or direct implantation or injection into a specific body part. The amount of HSP polypeptide contained in the sustained release system can depend, for example, on where the composition is to be administered, the kinetics and duration of the release of the HSP polypeptides provided herein, as well as the nature of the condition, disorder and/or disease to be treated and/or cared for.

The HSP polypeptides provided herein also can be adsorbed on solid organic polymers or solid mineral supports including but not limited to, talc, bentonite, silica, starch or maltodextrin, among others. The HSP polypeptides provided herein, and compositions thereof, also can be incorporated into fabrics, non-woven fabrics and medical devices which are in direct contact with the skin, thus releasing the HSP polypeptides, whether by biodegradation of the binding system to the fabric, non-woven fabric or medical device, or by the friction between them and the body, due to body moisture, the skin's pH or body temperature. Furthermore, the fabrics and non-woven fabrics can be used for making garments that are in direct contact with the body. For example, the fabrics, non-woven fabrics and medical devices containing the HSP polypeptides provided herein are used for the treatment and/or care of those conditions, disorders and/or diseases, such as inflammatory disorders, which are improved or prevented by a stimulation of heat shock protein synthesis or by polypeptides that can reduce osmotic stress and/or have an osmoprotective effect.

Examples of fabrics, non-woven fabrics, garments, medical devices and means for immobilizing the peptides to them, among which are the delivery systems and/or the sustained release systems described above, are known to those of skill in the art. Examples of fabrics, non-woven fabrics, garments and medical devices include, but are not limited to, bandages, gauzes, t-shirts, socks, tights, underwear, girdles, gloves, diapers, sanitary napkins, dressings, bedspreads, wipes, adhesive patches, non-adhesive patches, occlusive patches, micro-electric patches and/or face masks.

The cosmetic or pharmaceutical compositions which contain the HSP polypeptides provided herein and/or their cosmetically or pharmaceutically acceptable salts, can be used in compositions of topical or transdermal application, optionally including cosmetically or pharmaceutically acceptable excipients necessary for formulating the desired administration form. In aspects, a composition provided herein includes one or more components chosen from an aqueous component, fatty component, volatile oil, non-volatile oil, surfactant, polymer, emulsifier, ultraviolet filter, sirtuin activator, anti-oxidant and a free-radical scavenger.

### Aqueous Component

A composition provided herein can, in certain aspects, include an aqueous component. In some embodiments, an aqueous component is present in an amount ranging from about 10% to about 99% by weight of the total weight of the composition. For example, an aqueous component may be present at about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, or about 95% by weight of the total weight of the composition. In some embodiments, an aqueous component is present in an amount ranging from about 20% to about 90% by weight, from about 50% to about 85% by weight, or from about 60% to about 75% by weight of the total weight of the composition.

In certain aspects, an aqueous component includes water. In aspects, an aqueous component includes at least one organic solvent miscible with water (at room temperature 25° C.). Organic solvents may include, for example, monoalcohols, polyols, glycol ethers, and mixtures thereof. Monoalcohols may include monoalcohols having from 2 to 6 carbon atoms (e.g., ethanol, isopropanol). Polyols may include polyols having from 2 to 20 carbon atoms, 2 to 10 carbon atoms, or 2 to 6 carbon atoms (e.g., glycerol, propylene glycol, butylene glycol, pentylene glycol, hexylene glycol, caprylylglycol, dipropylene glycol, diethylene glycol). Glycol ethers may include glycol ethers having from 3 to 16 carbon atoms (e.g., mono-, di- or tri-propylene glycol (C1-C4)alkyl ethers, mono-, di- or tri-ethylene glycol (C1-C4)alkyl ethers).

### Fatty Component

A composition provides herein can, in certain aspects, include one or more fatty components. Fatty components may include oils, waxes, fatty acids, fatty alcohols, and mixtures thereof. In aspects, a composition herein is in the form of an emulsion (e.g., an oil-in-water emulsion), and includes a dispersed fatty component containing at least one oil. The term oil generally refers to any fatty substance that is in liquid form at ambient temperature (20-25° C.) and at atmospheric pressure. In aspects, a composition herein is oil-free.

In certain aspects, a fatty component is present in an amount ranging from about 1% to about 30% by weight of the total weight of the composition. For example, a fatty component may be present at about 2% by weight, about 5% by weight, about 10% by weight, about 15% by weight, about 20% by weight, or about 25% by weight of the total weight of the composition. In aspects, a fatty component is present in an amount ranging from about 2% to about 20% by weight, or about 3% to about 15% by weight of the total weight of the composition.

An oil herein may be chosen from volatile and non-volatile oils of hydrocarbon-based, silicone or fluoro type. An oil may be of animal, vegetable, mineral or synthetic origin. The term hydrocarbon-based oil generally refers to an oil formed essentially of, or consisting of, carbon and hydrogen atoms, may include oxygen and nitrogen atoms, and generally contains no silicon or fluorine atoms. A hydrocarbon-based oil may contain ester, ether, amine and/or amide groups. The term silicone oil generally refers to an oil containing at least one silicon atom, and may contain one or more Si—O groups. The term fluoro oil generally refers to an oil containing at least one fluorine atom.

In certain aspects, a composition herein includes a fatty alcohol. Fatty alcohols may have the structure R—OH, where R is chosen from saturated and unsaturated, linear and branched radicals containing for example, 4 to 40 carbon atoms, 6 to 30 carbon atoms, or 12 to 20 carbon atoms. In certain aspects, R may be chosen from C12-C20 alkyl and C12-C20 alkenyl groups. R may or may not be substituted with at least one hydroxyl group. Non-limiting examples of fatty alcohols include lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

In certain aspects, a composition herein includes a fatty acid. Fatty acids may include, but are not limited to, for example, carboxylic acids, saturated or unsaturated, having for example, 6 to 30 carbon atoms, or 9 to 30 carbon atoms. Fatty acids may be chosen from among the following examples including, but not limited to, myristic acid, palmitic acid, stearic acid, behenic acid, oleic acid, linoleic acid, linolenic acid and isostearic acid.

### Volatile Oil

The term volatile oil generally refers to an oil (or non-aqueous medium) capable of evaporating on contact with the skin in less than one hour, at ambient temperature and atmospheric pressure. A volatile oil is a volatile cosmetic oil that is liquid at ambient temperature, having in particular a non-zero vapor pressure, at ambient temperature and atmospheric pressure, in particular having a vapor pressure ranging from 0.13 Pa to 40,000 Pa (10-3 to 300 mmHg), 1.3 Pa to 13,000 Pa (0.01 to 100 mmHg), or 1.3 Pa to 1300 Pa (0.01 to 10 mmHg). A volatile oil generally has a boiling point, measured at atmospheric pressure, ranging from about 150° C. to about 260° C., or ranging from about 170° C. to about 250° C.

A volatile oil may be a hydrocarbon-based or silicone oil. In aspects, a hydrocarbon-based volatile oil may be chosen from hydrocarbon-based oils having a flash point ranging from about 40° C. to about 102° C., about 40° C. to about 55° C., or about 40° C. to about 50° C.

Hydrocarbon-based volatile oils may contain 8 to 16 carbon atoms, which may be linear or branched, and mixtures thereof. Branched hydrocarbon-based volatile oils may include C8-C16 alkanes, such as C8-C16 isoalkanes (also referred to as isoparaffins), isododecane, isodecane, isohexadecane, an undecane/tridecane mixture, dodecane, tetradecane, and mixtures thereof; and C8-C16 branched esters, such as isohexyl neopentanoate, and mixtures thereof.

### Non-Volatile Oil

A non-volatile oil may be chosen from carbon-based, hydrocarbon-based, silicone oils, and fluoro oils of mineral, animal, vegetable or synthetic origin, and mixtures thereof. For example, non-volatile hydrocarbon-based oils may include liquid paraffin or liquid petroleum jelly, isoeicosane, soya oil, perhydrosqualene, sweet almond oil, beauty-leaf oil, palm oil, grapeseed oil, sesame oil, maize oil, rapeseed oil, sunflower oil, cottonseed oil, apricot oil, castor oil, avocado oil, jojoba oil, olive oil or cereal germ oil; esters of lanolic acid, of oleic acid, of la uric acid, of stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate, lactate, 2-diethylhexyl succinate, diisostearyl malate, glyceryl triisostearate, diglyceryl triisostearate; carbonates, such as dicaprylyl carbonate; ethers, such as dicaprylyl ether; higher fatty acids, such as myristic acid, palmitic acid, stearic acid, behenic acid, oleic acid, linoleic acid, linolenic acid, isostearic acid; higher fatty alcohols, such as cetanol, stearyl alcohol, oleyl alcohol, linoleyl alcohol, linolenyl alcohol, isostearyl alcohol, octyldodecanol, and the like.

In certain aspects, a composition herein is a transparent emulsion that can include at least one oil selected from liquid esters of saturated or unsaturated, linear or branched C1-C26 aliphatic monoacids or polyacids and of saturated or unsaturated, linear or branched C1-C26 aliphatic monoalcohols or polyalcohols, the total number of carbon atoms of the esters being greater than or equal to 10. In certain aspects, for the esters of monoalcohols, at least one from among the alcohol and the acid from which the esters are derived is branched. Monoesters of monoacids and of monoalcohols may include, for example, ethyl palmitate, ethyl hexyl palmitate, isopropyl palmitate, dicaprylyl carbonate, alkyl myristates such as isopropyl myristate and ethyl myristate, isocetyl stearate, 2-ethylhexyl isononanoate, isononyl isononanoate, isodecyl neopentanoate, and isostearyl neopentanoate.

Surfactants and/or Emulsifiers

A composition provided herein can, in certain aspects, include one or more surfactants and/or emulsifiers. For example, a composition herein may include one or more surfactants to obtain a transparent emulsion (e.g., a transparent oil-in-water emulsion). A surfactant may be present in a composition herein in an amount ranging from 0.01% by weight to about 10% by weight, from about 0.5% by weight to about 7% by weight, or from about 1% by weight to about 5% by weight, relative to the total weight of the composition. Surfactants may include nonionic surfactants, anionic surfactants, and mixtures thereof.

A surfactant may include, for example, a fatty acid ester of polyethylene glycol and/or a glyceryl ester as a nonionic surfactant. Examples of fatty acid ester of polyethylene glycol may include PEG-8 Stearate, PEG-6 Oleate, PEG-6 Isostearate, PEG-12 Isostearate, PEG-12 Diisostearate, PEG-8 Isostearate, PEG-8 Diisostearate, PEG-10 Isostearate, PEG-100 stearate, and the like. Examples of glyceryl ester may include glyceryl oleate, glyceryl monostearate (or glyceryl stearate), glyceryl monoisostearate, glyceryl monopalmitate, glyceryl monobehenate, and mixtures thereof.

In certain aspects, a composition herein can include one or more nonionic surfactants. Non-limiting examples of nonionic surfactants include alkyl- and polyalkyl-esters of glycerol, polyglycerol ester of fatty acids, mixtures of alkyl- and polyalkyl-esters of glycerol with polyglyceryl, such as polyglyceryl-3 methylglucose distearate, oxyalkylenated (e.g., polyoxyethylenated), fatty acid esters of glycerol; oxyalkylenated fatty acid esters of sorbitan; oxyalkylenated (oxyethylenated and/or oxypropylenated) fatty acid esters (esters of polyethylene glycol and fatty acids); oxyalkylenated (oxyethylenated and/or oxypropylenated) fatty alcohol ethers; sugar esters, for instance sucrose stearate; fatty alcohol ethers of sugars, especially alkyl polyglucosides (APGs) such as decyl glucoside, lauryl glucoside, cetostearyl glucoside (e.g., as a mixture with cetostearyl alcohol), and arachidyl glucoside (e.g., in the form of a mixture of arachidyl alcohol), behenyl alcohol, arachidyl glucoside, lecithins and derivatives (e.g., biophilic), sugar esters, and sodium stearoyl lactylate.

In aspects, a composition provided herein includes one or more anionic surfactants. Non-limiting examples of anionic surfactants include glyceryl stearate, PEG-100 stearate, Poloxamer 338, alkylamido ether sulfates, alkylaryl polyether sulfates, monoglyceride sulfates, sultanates, such as alkylsulfonates, alkylamide sultanates, alkylarylsulfonates, alpha-olefin sultanates, paraffin sultanates, sulfosuccinates, alkylsulfosuccinates, alkyl ether sulfosuccinates, alkylamide sulfosuccinates, alkyl sulfoacetates, acylsarcosinates, acylglutamates, alkylsulfosuccinamates. N-acyl N-methyltaurates, N-acylisethionates, N-acyltaurates, salts of alkyl monoesters and polyglycoside-polycarboxylic acids, acyllactylates, mixed esters of organic acids with glycerol, such as glyceryl stearate citrate and as glyceryl stearate lactate, salts of D-galactoside uronic acids, salts of alkyl ether carboxylic acids, salts of alkyl aryl ether carboxylic acids, and salts of alkylamido ether carboxylic acids; or the non-salified forms of the above compounds, the alkyl and acyl groups the above compounds containing from 6 to 24 carbon atoms and the aryl group denoting a phenyl group. Some of the above compounds may be oxyethylenated and may contain from 1 to 50 ethylene oxide units.

Anionic surfactants also may include anionic derivatives of proteins of vegetable origin or of silk proteins, phosphates and alkyl phosphates, carboxylates, sulphosuccinates, amino acid derivatives, alkyl sulphates, alkyl ether sulphates, sulphonates, isethionates, taurates, alkyl sulphoacetates, polypeptides, anionic derivatives of alkyl poly glucosides, and mixtures thereof.

Emulsifiers that can be used in the compositions provided herein may include non-ionic or ionic emulsifiers (anionic, cationic or amphoteric). In aspects, non-ionic emulsifiers can include polyalkylene glycol ethers of fatty alcohols containing from 8 to 30 carbon atoms, e.g., from 10 to 22 carbon atoms; alkyl esters of polyoxyalkylenated sorbitan and polyoxyethylene, wherein the alkyl radical includes between 8 to 30 carbon atoms, e.g., from 10 to 22 carbon atoms; polyoxyalkylene alkyl esters and polyoxyethylene, wherein the alkyl radical includes between 8 to 30 carbon atoms, e.g., 10 to 22 carbon atoms; polyethylene glycols; polypropylene glycols; diethylene glycols; and mixtures thereof. In aspects, the emulsifier is polysorbate 20, ceteareth 20, diutan gum, carrageenan, gellan gum, welan gum, pectin, sclerotium gum, starch, or galactoarabinan. In certain aspects, the emulsifier is xanthan gum. The amount of emulsifier or emulsifiers is generally from 0.001% to 30% by weight, based on the total weight of the composition. In certain aspects, the emulsifier is present at greater than zero percent and less than about 2% by weight. In aspects, one or more emulsifiers are present at greater than zero percent and less than 1%, e.g., about 0.2%.

### Polymer

A composition provided herein can, in aspects, include one or more polymers. Suitable polymers include, but are not limited to, polylactic acid (PLA), poly C10-C30 alkyl acrylate, acrylates/C10-C30 alkyl acrylate crosspolymer, styrene/acrylates copolymer, lauryl methacrylate/glycol dimethacrylate crosspolymer, ammonium acryloyldimethyltaurate/vp copolymer, dimethicone/vinyl dimethicone crosspolymer, ammonium polyacryloyldimethyl taurate, aluminum starch octenylsuccinate and mixtures thereof.

### Additive

A composition provided herein can, in aspects, include one or more additives. For example, a composition may include one or more fragrances, vitamins (e.g., tocopherol, niacinamide, vitamin B3, vitamin B6), NAD-boosting compounds. (e.g. nicotinamide riboside (NR), nicotinamide mononucleotide (NMN), C6-C18 fatty acid nicotinate esters such as myristyl nicotinate or tetradecyl nicotinate), NAMPT inhibitors, preservatives (e.g., phenoxyethanol and salicylic acid), silicones (e.g., dimethicone, caprylyl methicone, vinyl dimethicone/methicone silsesquioxane crosspolymer), fatty compounds, fillers (e.g., silicas (e.g., silica silylate), mica, magnesium oxide, nylon-12, nylon-66, cellulose, talc, talc and methicone, talc and dimethicone, perlite, sodium silicate, pumice, PTFE, polymethyl methacrylate, alumina, calcium sodium borosilicate, magnesium carbonate), solvents (e.g., short-chain alcohols (e.g., ethanol), glycols, polyols, glycerin, caprylyl glycol, pentylene glycol, propylene glycol, butylene glycol), thickeners, organic or mineral additives, physical and/or chemical sunscreens, sequestering agents, antioxidants, insoluble active agents, liposoluble active agents, water-soluble active agents, moisturizers such as polyols (e.g., glycerol), pH adjusters (acids or bases), additional active agents (e.g., agents extracted from plants, agents resulting from biotechnology, disodium EDTA, triethanolamine, capryloyl salicylic acid, hydroxyethylpiperazine ethane sulfonic acid), mineral active agents, and/or tensioning agents.

Additives may be present at concentrations ranging from about 0.1% to about 90% by weight, from about 0.1% to 10% by weight, from about 1% to about 90% by weight, from about 5% to about 80% by weight, from about 10% to about 70% by weight, from about 15% to about 60% by weight, or from about 20% by weight to about 50% by weight, based on the total weight of the composition herein.

In certain aspects, a composition provided herein includes one or more sirtuin activators. A sirtuin activator sometimes is a phenol or a stilbenoid. A non-limiting example of sirtuin activator is resveratrol (3,5,4′-trihydroxy-trans-stilbene).

In certain aspects, a composition provided herein includes one or more anti-oxidants. An anti-oxidant sometimes is a free-radical scavenger, and sometimes is a phenol or a stilbenoid. Non-limiting examples of anti-oxidants include vitamin A, vitamin E, butylated hydroxytoluene (BHT), Urolithin A. and butylated hydroxyanisole (BHA). In aspects, the anti-oxidant improves stability or shelf-life of the composition. In certain aspects, the anti-oxidant provides protection against damage to cells or surfaces on which the composition is applied.

### UV Filter

A composition provided herein can, in aspects, include one or more UV filter components. UV filters may be active in the UV-A and/or UV-B region. UV filters may be hydrophilic and/or lipophilic. UV filters may be solid or liquid. Any suitable UV filter may be included in a composition provided herein including, but not limited to, anthranilic compounds; dibenzoylmethane compounds; cinnamic compounds; salicylic compounds; camphor compounds; benzophenone compounds; β,β-diphenylacrylate compounds: triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinal; cysteine; aromatic amino acids; peptides having an aromatic amino acid residue; and mixtures thereof.

In certain aspects, a UV filter may be chosen from butyl methoxydibenzoylmethane, ethylhexyl methoxycinnamate, homosalate, octocrylene, phenylbenzimidazole sulfonic acid, benzophenone-3, benzophenone-4, benzophenone-5, n-hexyl 2-(4-diethylamino-2-hydroxybenzoyl)benzoate, 1,r-(1,4-piperazinediyl)bis[1-[2-[4-(diethylamino)-2-hydroxybenzoyl]phenyl]-methanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone. 2,4,6-tris(dineopentyl 4′-aminobenzalmalonate)-s-triazine, 2,4,6-tris(diisobutyl 4′-aminobenzalmalonate)-s-triazine, 2,4-bis-(n-butyl 4′-aminobenzalmalonate)-6-[(3-{1,3,3,3-tetramethyl-1-[(trimethylsilyloxy]-disiloxanyl}propyl)amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxybenzalmalonate, 1,1-dicarboxy (2,2′-dimethylpropyl)-4,4-diphenylbutadiene, 2,4-bis [5-1 (dimethylpropyl)benzoxazol-2-yl-(4-phenyl)imino]-6-(2-ethylhexyl)imino-1,3,5-triazine, camphor benzylkonium methosulfate, and mixtures thereof.

In aspects, compositions provided herein further include one or more compounds selected from a benzophenone or derivative thereof, a cinnamate compound or derivative thereof, a salicylate compound or derivative thereof, a mineral-based compound or derivative thereof, oxybenzone, avobenzone, octinoxate, octisalate, octocrylene, homosalate, titanium dioxide and zinc oxide. In aspects, one or more or all of the following compounds are excluded: a benzophenone or derivative thereof, a cinnamate compound or derivative thereof, a salicylate compound or derivative thereof, a mineral-based compound or derivative thereof, oxybenzone, avobenzone, octinoxate, octisalate, octocrylene, homosalate, titanium dioxide and zinc oxide. In various implementations, one or more or all of the following compounds are either not present, or are present in an amount that does not provide for UV protection: a benzophenone or derivative thereof, a cinnamate compound or derivative thereof, a salicylate compound or derivative thereof, a mineral-based compound or derivative thereof, oxybenzone, avobenzone, octinoxate, octisalate, octocrylene, homosalate, titanium dioxide and zinc oxide.

The compositions of topical or transdermal application can be produced in any solid, liquid or semi-solid formulation, including but not limited to, creams, emulsions including but not limited to, oil and/or silicone in water emulsions, water-in-oil and/or silicone emulsions, water/oil/water or water/silicone/water type emulsions, and oil/water/oil or silicone/water/silicone type emulsions, anhydrous compositions, aqueous dispersions, oils, milks, balsams, foams, lotions, gels, cream gels, hydroalcoholic solutions, hydroglycolic solutions, hydrogels, liniments, sera, soaps, shampoos, conditioners, serums, polysaccharide films, ointments, mousses, pomades, powders, bars, pencils and sprays or aerosols (sprays), including leave-on and rinse-off formulations. These topical or transdermal application formulations can be incorporated using techniques known to those of skill in the art into different types of solid accessories including but not limited to, bandages, gauzes, t-shirts, socks, tights, underwear, girdles, gloves, diapers, sanitary napkins, dressings, bedspreads, wipes, adhesive patches, non-adhesive patches, occlusive patches, micro-electric patches or face masks, or they can be incorporated into different make-up products such as make-up foundation, such as fluid foundations and compact foundations, make-up removal lotions, make-up removal milks, under-eye concealers, eye shadows, lipsticks, lip protectors, lip gloss and powders, among others. In certain aspects, a composition provided herein is in the form of a cream, lotion, emulsion, oil, butter, paste, balm, stick, foam, gel, serum, ointment, mousse, powder, semi-solid formulation, spray or aerosol. In certain aspects, a composition provided herein is in the form of sunscreen, sunblock, body moisturizer, facial moisturizer, hair moisturizer, make-up foundation, lipstick, lip balm, hair spray, or hair dye. In various embodiments, the composition is in the form of a solution, dispersion, suspension, emulsion, or colloid. In aspects, the composition is in the form of a cream, lotion, paste, oil, foam, gel, serum, powder, spray or aerosol.

The cosmetic and pharmaceutical compositions provided herein can include agents that increase the percutaneous absorption of the HSP polypeptides provided herein, including but not limited to, dimethylsulfoxide, dimethylacetamide, dimethylformamide, surfactants, azone (1-dodecylazacycloheptane-2-one), alcohol, urea, ethoxydiglycol, acetone, propylene glycol or polyethylene glycol, among others. Furthermore, the cosmetic or pharmaceutical compositions of the HSP polypeptides provided herein can be applied to local areas to be treated by means of iontophoresis, sonophoresis, electroporation, microelectric patches, mechanical pressure, osmotic pressure gradient, occlusive cure, microinjections or needle-free injections by means of pressure, such as injections by oxygen pressure, or any combination thereof, to achieve a greater penetration of the HSP polypeptides provided herein. The application area can be determined by the nature of the condition, disorder and/or disease to be treated and/or cared for.

In certain aspects, the cosmetic or pharmaceutical compositions containing the HSP polypeptides provided herein, or their cosmetically or pharmaceutically acceptable salts, can be used in different types of formulations for oral administration, e.g., in the form of oral cosmetics and pharmaceutical drugs, including but not limited to, capsules, including gelatin capsules, soft capsules, hard capsules, tablets, including sugar coated tablets, powders, granules, chewing gum, solutions, suspensions, emulsions, syrups, polysaccharide films, jellies or gelatins, and any other form known to those of skill in the art. In aspects, the HSP polypeptides provided herein can be incorporated into any form of functional food or fortified food, including but not limited to, dietary bars or compact or non-compact powders. In aspects, these powders can be dissolved, for example, in water, juices, soda, dairy products, soya derivatives or can be incorporated into dietary bars. The HSP polypeptides provided herein can, in aspects, be formulated with common excipients and adjuvants for oral compositions or food supplements, including but not limited to, fat components, aqueous components, humectants, preservatives, texturizing agents, flavors, aromas, antioxidants and colorants common in the food industry.

In aspects, cosmetic or pharmaceutical compositions containing the HSP polypeptides provided herein and/or their cosmetically or pharmaceutically acceptable salts can also be administered by topical or transdermal route, as well as by any other appropriate route, as for example oral or parenteral route, for which they will include the pharmaceutically acceptable excipients necessary for the formulation of the desired administration form. As used herein, the term “parenteral,” as used herein, includes nasal, auricular, ophthalmic, rectal, urethral, vaginal, subcutaneous, intradermal, intravascular injections, such as intravenous, intramuscular, intraocular, intravitreous, intracorneal, intraspinal, intramedullary, intracranial, intracervical, intracerebral, intrameningeal, intraarticular, intrahepatic, intrathoracic, intratracheal, intrathecal and intraperitoneal, and any another similar injection or infusion technique.

In certain aspects, the polypeptides, compositions or combinations provided herein are administered along with or are co-formulated with one or more additional active agents including, but not limited to, an additional agent for disorders and/or diseases of the skin, mucous membranes and/or hair, a chemotherapeutic agent, an analgesic agent, an antibiotic, an anti-inflammatory agent, an antimicrobial agent, an amoebicidal agent, a trichomonacidal agent, an anti-Parkinson agent, an anti-malarial agent, an anticonvulsant agent, an anti-depressant agent, and antiarthritics agent, an anti-fungal agent, an antiviral agent, an antihypertensive agent, an antipyretic agent, an anti-parasite agent, an antihistamine agent, an alpha-adrenergic agonist agent, an alpha blocker agent, an anesthetic agent, a bronchial dilator agent, a biocide agent, a bactericide agent, a bacteriostat agent, a beta adrenergic blocker agent, a calcium channel blocker agent, a cardiovascular drug agent, a contraceptive agent, a decongestant agent, a diuretic agent, a depressant agent, a diagnostic agent, a electrolyte agent, a hypnotic agent, a hormone agent, a hyperglycemic agent, a muscle relaxant agent, a muscle contracting agent, an ophthalmic agent, a parasympathomimetic agent, a psychic energizer agent, a sedative agent, a sympathomimetic agent, a tranquilizer agent, an urinary agent, a vaginal agent, a viricide agent, a vitamin agent, a non-steroidal anti-inflammatory agent, an angiotensin converting enzyme inhibitor agent, a polypeptide, a protein, a nucleic acid, a drug, an organic molecule or a sleep inducer. In some aspects, the additional active agent is an agent that reduces osmotic stress, or is osmoprotective (e.g., prevents osmotic stress, such as hyperosmotic stress). In aspects, the additional active agent that reduces osmotic stress, or is osmoprotective can include, but is not limited to, sorbitol, myoinositol, a heat shock protein other than the HSP polypeptides provided herein, or an inhibitor of aldose reductase (AR), e.g., fidarestat. In some aspects, the aforementioned additional active agents are independently formulated and are administered sequentially, serially, simultaneously, concurrently or intermittently with the compositions or combinations provided herein.

In aspects, among the cosmetically or pharmaceutically acceptable adjuvants contained in the cosmetic or pharmaceutical compositions provided herein are additional ingredients commonly used in compositions for the treatment and/or care of the skin, mucous membranes and/or hair including but not limited to, heat shock proteins other than the HSP polypeptides provided herein, heat shock protein synthesis stimulating agents, acetylcholine-receptor aggregation inhibitors, muscle contraction inhibiting agents, anticholinergic agents, elastase inhibiting agents, matrix metalloproteinase inhibiting agents, melanin synthesis stimulating or inhibiting agents, whitening or depigmenting agents, propigmenting agents, self-tanning agents, anti-aging agents, NO-synthase inhibiting agents, 5a-reductase inhibiting agents, lysyl- and/or prolyl hydroxylase inhibiting agents, antioxidants, free radical scavengers and/or agents against atmospheric pollution, reactive carbonyl species scavengers, anti-glycation agents, antihistamine agents, antiviral agents, antiparasitic agents, emulsifiers, emollients, organic solvents, liquid propellants, skin conditioners such as humectants, substances that retain moisture, alpha hydroxyacids, beta hydroxyacids, moisturizers, epidermal hydrolytic enzymes, vitamins, amino acids, proteins, pigments or colorants, dyes, gelling polymers, thickeners, surfactants, softening agents, anti-wrinkle agents, agents able to reduce or treat bags under the eyes, exfoliating agents, antimicrobial agents, antifungal agents, fungistatic agents, bactericidal agents, bacteriostatic agents, agents stimulating the synthesis of dermal or epidermal macromolecules and/or capable of inhibiting or preventing their degradation, such as for example collagen synthesis-stimulating agents, elastin synthesis-stimulating agents, decorin synthesis-stimulating agents, laminin synthesis-stimulating agents, defensin synthesis-stimulating agents, aquaporin synthesis-stimulating agents, hyaluronic acid synthesis-stimulating agents, fibronectin synthesis-stimulating agents, sirtuin synthesis-stimulating agents, agents stimulating the synthesis of lipids and components of the stratum corneum (ceramides, fatty acids, etc.), agents that inhibit collagen degradation, other agents that inhibit elastin degradation, agents that inhibit serine proteases such as cathepsin G, agents stimulating fibroblast proliferation, agents stimulating keratinocyte proliferation, agents stimulating adipocyte proliferation, agents stimulating melanocyte proliferation, agents stimulating keratinocyte differentiation, agents stimulating adipocyte differentiation, agents that inhibit acetylcholinesterase, skin relaxant agents, glycosaminoglycan synthesis-stimulating agents, antihyperkeratosis agents, comedolytic agents, antipsoriasis agents, DNA repair agents, DNA protecting agents, stabilizers, anti-itching agents, agents for the treatment and/or care of sensitive skin, firming agents, anti-stretch mark agents, binding agents, agents regulating sebum production, lipolytic agents or agents stimulating lipolysis, anti-cellulite agents, antiperspirant agents, agents stimulating healing, coadjuvant healing agents, agents stimulating re-epithelialization, coadjuvant re-epithelialization agents, cytokine growth factors, calming agents, anti-inflammatory and/or analgesic agents, anesthetic agents, agents acting on capillary circulation and/or microcirculation, agents stimulating angiogenesis, agents that inhibit vascular permeability, venotonic agents, agents acting on cell metabolism, agents to improve dermal-epidermal junction, agents inducing hair growth, hair growth inhibiting or retardant agents, hair loss retardant agents, preservatives, perfumes, chelating agents, vegetable extracts, essential oils, marine extracts, agents obtained from a bio-fermentation process, mineral salts, cell extracts and sunscreens (organic or mineral photoprotective agents active against ultraviolet A and/or B rays) among others, provided they are physically and chemically compatible with the other components of the composition, including the HSP polypeptides provided herein. The additional ingredients can be synthetic or natural, such as vegetable extracts, or obtained by a biotechnological process or a combination of a synthetic process and a biotechnological process, or any such additional ingredients or equivalents thereof as known to those of skill in the art. As used herein, biotechnological process is understood to be any process which produces the active ingredient, or part of it, in an organism, or in a part of it.

Also provided herein, in aspects, are cosmetic or pharmaceutical compositions containing a cosmetically or pharmaceutically effective amount of at least one HSP polypeptide provided herein and/or its cosmetically or pharmaceutically acceptable salts, and additionally a cosmetically or pharmaceutically effective amount of at least one extract, synthetic compound or bio-fermentation product that stimulates heat shock protein synthesis, including, but not limited to, extracts of Opuntia ficus indica, Salix alba, Lupinus spp., Secale cereale, extracts of red algae of the genus Porphyra, extracts of crustaceans of the genus Artemia, jojoba seed oil, grape seed extracts, green tea extracts, geranylgeranylacetone, celastrol, zinc and its salts, 2-cyclopenten-1-one, proteasome inhibitors including but not limited to, bortezomib; prostaglandins and their derivatives, hydroxylamine and its derivatives including but not limited to, bimoclomol; chalcone and its derivatives, hyperosmotic agents including but not limited to, sorbitol and its derivatives, mannitol and its derivatives or glycerol and its derivatives, isosorbide, urea or salicylic acid and its derivatives among others, or mixtures thereof.

Also provided herein, in aspects, are cosmetic or pharmaceutical compositions containing a cosmetically or pharmaceutically effective amount of at least one HSP polypeptide provided herein and/or its cosmetically or pharmaceutically acceptable salts, and additionally a cosmetically or pharmaceutically effective amount of at least one extract that is an anti-wrinkle agent and/or anti-aging agent including, but not limited to, the extracts of Vitis vinifera, Rosa canina, Curcuma longa, Iris pallida, Theobroma cacao, Ginkgo biloba, Leontopodium Alpinum or Dunaliella salina among others or, in addition, at least one synthetic compound or bio-fermentation product which is an anti-wrinkle agent and/or an anti-aging agent including but not limited to Matrixyl® [INCI: Palmitoyl Pentapeptide-4], Matrixyl 3000® [INCI: Palmitoyl Tetrapeptide-7, Palmitoyl Oligopeptide], Essenskin™ [INCI: calcium hydroxymethionine], Renovage [INCI: teprenone] or Dermaxyl® [INCI: Palmitoyl Oligopeptide] marketed by Sederma, Vialox® [INCI: Pentapeptide-3], Syn®-Ake® [INCI: Dipeptide Diaminobutyroyl Benzylamide Diacetate], Syn®-Coll [INCI: Palmitoyl Tripeptide-5], Phytaluronate [INCI: Locust Bean (Ceratonia Siliqua) Gum] or Preregen® [INCI: Glycine Soja (Soybean) Protein, Oxido Reductases] marketed by Pentapharm/DSM, Myoxinol™ [INCI: Hydrolyzed Hibiscus Esculentus Extract], Syniorage™ [INCI: Acetyl Tetrapeptide-1 1], Dermican™ [INCI: Acetyl Tetrapeptide-9] or DN-AGE™ LS [INCI: Cassia Alata leaf Extract] marketed by Laboratoires Serobiologiques/Cognis, CR [INCI: Methylsilanol Mannuronate] or Hydroxyprolisilane CN® [INCI: Methylsilanol Hydroxyproline Aspartate] marketed by Exsymol, Argireline® [INCI: Acetyl Hexapeptide-8], SNAP-7 [INCI: Acetyl Heptapeptide-4], SNAP-8 [INCI: Acetyl Octapeptide-3], Leuphasyl® [INCI: Pentapeptide-18], Inyline™ [INCI proposal: Acetyl Hexapeptide-25], Aldenine@ [INCI: Hydrolized wheat protein, hydrolized soy protein, Tripeptide-1], Preventhelia™ [INCI: Diaminopropionoyl Tripeptide-33], Decorinyl® [INCI: Tripeptide-10 Citrulline], Trylagen® [INCI: Pseudoalteromonas Ferment Extract, Hydrolyzed Wheat Protein, Hydrolyzed Soy Protein, Tripeptide-10 Citrulline, Tripeptide-1], Eyeseryl® [INCI: Acetyl Tetrapeptide-5], Peptide AC29 [INCI: Acetyl Tripeptide-30 Citrulline], Relistase™ [INCI proposal: Acetyl Tetrapeptide-30], Lipochroman-6 Dimethylmethoxy Chromanol], Chromabright™ [INCI: Dimethylmethoxy Chromanyl Palmitate], Antarcticine® [INCI: Pseudoalteromonas Ferment Extract] or Vilastene™ [INCI: Lysine HCl, Lecithin, Tripeptide-10 Citrulline] marketed by Lipotec. Kollaren® [INCI: Tripeptide-1, Dextran] marketed by Institut Europeen de Biologie Cellulaire, Collaxyl® IS [INCI: Hexapeptide-9], Laminixyl IST [INCI: Heptapeptide], Orsirtine™ GL [INCI: Oryza Sativa (Rice) Extract], D'Orientine™ IS [INCI: Phoenix Dactylifera (Date) Seed Extract], Phytoquintescine™ [INCI: Einkorn (Triticum Monococcum) Extract] or Quintescine™ IS [INCI: Dipeptide-4] marketed by Vincience/ISP, BONT-L-Peptide [INCI: Palmitoyl Hexapeptide-19] marketed by Infinitec Activos, Deepaline™ PVB [INCI: Palmitoyl hydrolyzed Wheat Protein] or Sepilift® DPHP [INCI: Dipalmitoyl Hydroxyproline] marketed by Seppic, Gatuline® Expression [INCI: Acmella oleracea Extract], Gatuline® In-Tense [INCI: Spilanthes Acmella Flower Extract] or Gatuline® Age Defense 2 [INCI: Juglans Regia (Walnut) Seed Extract] marketed by Gattefosse, Thalassine™ [INCI: Algae Extract] marketed by Biotechmarine, ChroNOline™ [INCI: Caprooyl Tetrapeptide-3] or Thymulen-4 [INCI: Acetyl Tetrapeptide-2] marketed by Atrium Innovations/Unipex Group, EquiStat [INCI: Pyrus malus Fruit Extract, Glycine Soja Seed Extract] or Juvenesce [INCI: Ethoxydiglicol and Caprylic Triglycerid, Retinol, Ursolic Acid, Phytonadione, Ilomastat] marketed by Coletica/Engelhard/BASF, Ameliox [INCI: Carnosine, Tocopherol, Silybum Marianum Fruit Extract] or PhytoCellTec Malus Domestica [INCI: Malus Domestica Fruit Cell Culture] marketed by Mibelle Biochemistry, Bioxilift [INCI: Pimpinella anisum Extract] or SMS Anti-Wrinkle® [INCI: Annona squamosa Seed Extract] marketed by Silab, antagonists of the Ca²⁺ channel including but not limited to, alverine, manganese or magnesium salts, certain secondary or tertiary amines, retinol and its derivatives, idebenone and its derivatives, Coenzyme Q10 and its derivatives, boswellic acid and its derivatives, GHK and its derivatives and/or salts, carnosine and its derivatives, DNA repair enzymes including, but not limited to, photolyase, T4 endonuclease V, or chloride channel agonists among others.

Also provided herein, in aspects, are cosmetic or pharmaceutical compositions that contain a cosmetically or pharmaceutically effective amount of at least one HSP polypeptide provided herein and/or its cosmetically or pharmaceutically acceptable salts, and, in addition, a cosmetically or pharmaceutically effective amount of at least one extract or combination of extracts that stimulates healing and/or re-epithelialization or coadjuvants of healing and/or re-epithelialization including but not limited to, the extracts of Centella asiatica, Rosa moschata, Echinacea angustifolia, Symphytum officinal, Equisetum arvense, Hypericum perforatum, Mimosa tenuiflora, Aloe vera, Polyplant® Epithelizing [INCI: Calendula officinalis, Hypericum perforatum, Chamomilla recutita, Rosmarinus officinalis] marketed by Provital, Cytokinol® LS 9028 [INCI: Hydrolyzed Casein, Hydrolyzed Yeast Protein, Lysine HCl] marketed by Laboratories Serobiologiques/Cognis or Deliner® [INCI: Zea mays (Corn) Kernel Extract] marketed by Coletica/Engelhard/BASF among others, and/or a cosmetically or pharmaceutically effective amount of at least one synthetic compound, extract or bio-fermentation product which stimulates healing and/or re-epithelialization including but not limited to, cadherins, integrins, selectins, hyaluronan acid receptors, immunoglobulins, fibroblast growth factor, connective tissue growth factor, platelet-derived growth factor, vascular endothelial growth factor, epidermal growth factor, insulin-like growth factor, keratinocyte growth factors, colony-stimulating factors, transforming growth factor-beta, tumor necrosis factor-alpha, interferons, interleukins, matrix metalloproteinases, protein tyrosine phosphatase receptors, Antarcticine® [INCI: Pseudoalteromonas Ferment Extract] or Decorinyl® [INCI: Tripeptide-10 Citrulline], marketed by Lipotec, among others, or a mixture thereof.

Also provided herein, in aspects, are cosmetic or pharmaceutical compositions that contain a cosmetically or pharmaceutically effective amount of at least one HSP polypeptide provided herein and/or its cosmetically or pharmaceutically acceptable salts, and, in addition, a cosmetically or pharmaceutically effective amount of at least one extract or combination of extracts delaying hair loss or inducing hair growth including but not limited to, extracts of Tussilago farfara or Achillea millefolium, and/or a cosmetically or pharmaceutically effective amount of at least one compound delaying hair loss or inducing hair growth, including but not limited to, nicotinic acid esters such as alkyl nicotinates C3-C₆ such as methyl or hexyl nicotinate, benzyl nicotinate, or tocopherol nicotinate; steroid and nonsteroidal anti-inflammatory agents, including but not limited to, hydrocortisone, its salts and derivatives or niflumic acid; retinoids including, but not limited to, all-trans-retinoic acid or tretinoin, isotretinoin, retinol or vitamin A, and its derivatives, such as acetate, palmitate, propionate, motretinide, etretinate and zinc trans retinoate; antibacterial agents including but not limited to, macrolides, pyranosides, and tetracycline, erythromycin; antagonists of calcium channels including but not limited to, cinnarizine and diltiazem; hormones including but not limited to, estriol, its analogues or tyrosine, its analogues and/or its salts; antiandrogen agents including but not limited to, oxendolone, spironolactone or diethylstilbestrol; anti-radicals including but not limited to, dimethyl sulfoxide; esterified oligosaccharides including but not limited to, those described in EP 0211610; derivatives of hexasaccharide acids including but not limited to, glucose saccharide acid or those described in EP 0375388; glucosidase inhibitors including but not limited to, D-glucaro-1,5-lactam or those described in EP 0334586; glycosaminoglycan and proteoglycan inhibitors including but not limited to, L-galactono-1,4-lactone or those described in EP 0277428; tyrosine kinase inhibitors including but not limited to, 1-amide-1-cyano(3,4-dihydroxyphenyl)ethylene or those described in EP 0403238, diazoxides including but not limited to, 7-(acetylthio)-4′,5′-dihydrospiro[androst-4-ene-17,2′-(3H) furan]-3-one, 3-methyl-7-chloro [2H]-1,2,4-benzothiadiazine or spiroxazone 1,1-dioxide; phospholipids including but not limited to, lecithin; salicylic acid and its derivatives, hydroxyl carboxylic or keto carboxylic acids and their esters, lactones and their salts; anthraltn, eicosa-5,8,11-trienoic acids and their esters or amides or minoxidil and their derivatives among others, or mixtures thereof.

Also provided herein, in aspects, are cosmetic or pharmaceutical compositions that contain a cosmetically or pharmaceutically effective amount of at least one HSP polypeptide provided herein and/or its cosmetically or pharmaceutically acceptable salts, and, in addition, a cosmetically or pharmaceutically effective amount of at least one sunscreen including, but not limited to, anthranilates, cinnamates, salicylates, derivatives of dibenzoylmethane, derivatives of camphor, derivatives of triazine, derivatives of benzophenone, derivatives of 3,3-diphenylacrylate, derivatives of benzotriazole, derivatives of benzylmalonate, derivatives of benzimidazole, imidazolines, derivatives of benzoallyl, derivatives of the p-aminobenzoic acid, polymers and silicones, derivatives of alkyl styrenes, nanopigments of metallic oxides including but not limited to, titanium oxide or zinc oxide or filters based on carbon nanotubes among others, or mixtures thereof.

Also provided herein, in aspects, are cosmetic or pharmaceutical compositions that contain a cosmetically or pharmaceutically effective amount of at least one HSP polypeptide provided herein and/or its cosmetically or pharmaceutically acceptable salts, and, in addition, and, in addition, a cosmetically or pharmaceutically effective amount of at least one protein from the heat shock protein family, including but not limited to, Hsp70, including Hsp72 and Hsp73, Hsp60, Hsp27 or Hsp90, among others.

The polypeptides and compositions provided herein can be used in a variety of applications including, but not limited to, one or more of the following aspects:

The polypeptides and compositions provided herein are, in some aspects, for cosmetic use. In certain aspects, the polypeptides and compositions provided herein are for pharmaceutical use. In aspects, the polypeptides and compositions provided herein are for reducing osmotic stress in a cell. In aspects, the polypeptides and compositions provided herein are for preventing osmotic stress in a cell.

### Inflammatory Disorders and Conditions of the Skin, Mucous Membranes and Hair

In aspects, the HSP polypeptides, compositions and combinations (e.g., with active reagents/adjuvants in addition to the HSP polypeptides provided herein), can be used for cosmetic or therapeutic applications to improve or ameliorate inflammatory disorders (e.g., diseases or conditions associated with or caused by inflammation), or improve or ameliorate diseases/conditions of the skin, mucous membranes and hair. Examples of such disorders/conditions include, but are not limited to, the following:

### Inflammatory Disorders

A growing body of evidence implicates hyperosmotic stress as a potent inflammatory stimulus by triggering proinflammatory cytokine release and inflammation. Hyperosmotic stress is linked to many maladies, including acute and chronic, as well as local and systemic, inflammatory disorders.

### A. Eye Disease

Maintaining osmolarity is a key feature of ocular tissues. The eye is a fluid-filled organ, and osmolality plays an important role in eye health. The intraocular space separating the cornea and lens is filled with a fluid, aqueous humor. Vitreous humor is a gel-like fluid that fills the space between the lens and retina. The cornea is constantly exposed to the external environment and corneal hydration by means of tear secretion is required for its proper function. Two features of dry eye disease include an increase in tear osmolarity and ocular surface inflammation; tear hyperosmolarity serves as a key diagnostic for this disorder. Hyperosmolarity (hyperosmotic stress) represents a potent inflammatory stimulus, and a strong correlation exists between tear hyperosmolarity and the severity of dry eye syndrome.

It was found that exposure to hypertonic solutions elicits the secretion of the pro-inflammatory cytokines IL6 and IL8 from corneal epithelial cells in vitro. Further, production of IL1β, TNF, and IL8 are up-regulated after treatment with hyperosmolar media in primary human limbal epithelial cultures. EGFR transactivation and the MAPK14 kinase pathway appear to also be involved in the corneal epithelial cell response to hypertonicity. Topical administration of hypertonic saline solution to the eyes of mice resulted in elevated levels of IL1β, TNF, and matrix metallopeptidase 9 levels on their corneal surface. Under these same conditions, JNK, ERK, and MAPK14 signaling pathways were also activated in the corneas of the animals. These results, in combination with the presence of hyperosmotic tears in chronic dry eye patients, are consistent with hyperosmotic stress being an important contributory factor in this condition.

Further, subjects with diabetes often develop diabetic retinopathy, a low-grade chronic inflammatory disease that results in retinal damage and eventual blindness. More than 80% of these patients who have had the disease for >10 years have experienced some degree of retinopathy. Osmotic stress caused by sorbitol accumulation is believed to play a role in microvascular complications associated with diabetic retinopathy, as is high aldose reductase (AR) activity. Hyperosmotic stress and NFAT5 activation (a transcription factor activated under hyperosmotic stress) are potent inducers of AR expression and AR is responsible for the metabolism of glucose to sorbitol. Although sorbitol can prevent or reduce hyperosmotic stress, AR inhibition nonetheless can be used to prevent or reduce osmotic stress because elevated AR activity can be pro-inflammatory during oxidative stress as the result of AR-mediated metabolism of aldehydes to corresponding alcohols, which then mediate inflammatory signals.

In vitro studies have revealed that retinal pigment epithelial cells exposed to hypertonic conditions exhibit a >11-fold increase in AR mRNA expression. A large number of studies have linked the activity of AR to diabetic retinopathy, and AR inhibitors are currently being used therapeutically in the disorder. The observation that hyperosmolarity induces significant AR expression supports a possible link between hypertonicity and diabetic retinopathy.

### B. Diabetes

Diabetes has many clinical manifestations including neuropathy, retinopathy, nephropathy, and hypertension. Significant increases in blood glucose contribute to plasma hyperosmolarity. A cross-sectional study revealed that elevated plasma osmolarity positively correlated with impaired glucose tolerance and diabetes. Subjects exhibiting serum hyperglycemia (elevated blood glucose) and electrolyte hypertonicity (elevated blood sodium and potassium) were found to be more than four times more likely to develop diabetes than subjects with hyperglycemia only. This observation suggests that elevated plasma tonicity is an important contributing factor to disease progression.

Vasopressin is released in response to high plasma osmolarity. Subjects with diabetes insipidus exhibit decreased release of vasopressin and/or decreased renal sensitivity to vasopressin, leading to an inability to respond to systemic hyperosmotic stress. Hyperglycemia is also linked with negative outcomes in clinical conditions including myocardial infarction and postoperative complications, such as increased risk of infection, and hyperosmotic stress also has been shown to inhibit interferon-γ (IFN-γ) expression in blood lymphocytes.

Given the important role of IFN in mounting an immune response to both viral and intracellular bacterial infections, hyperosmotic stress may contribute to the increased susceptibility to infection observed in patients with hyperglycemia. In vitro studies suggest that elevated glucose levels enhance inflammation during infection by means of hyperosmotic stress-induced cytokine release. Studies using in vivo mouse models of diabetic cataract formation identified both oxidative stress (as a result of glucose metabolism) and osmotic stress as contributing to the pathology. It has also been suggested that chronic oxidative stress associated with glucose metabolism impairs osmoregulatory pathways, thereby hindering a tissue's ability to respond to osmotic stress.

Hyperosmotic stress may also be associated with insulin resistance. In skeletal myocytes and adipocytes, insulin regulates blood glucose levels by stimulating translocation of SLC2A4 glucose transporters (also known as Glut4) to the plasma membrane, leading to increased glucose transport into the cell. Increases in extracellular non-glucose solute concentrations also can cause a corresponding increase in glucose uptake by adipocytes and myocytes. Insulin receptors belong to a subfamily of the tyrosine kinase receptor protein family. The binding of insulin results in activation of receptor kinase activity and phosphorylation of cellular proteins. Insulin receptor-mediated tyrosine phosphorylation of insulin receptor substrate-1 (IRS1) promotes the binding and activation of phosphoinositide 3 (PI3)-kinase. PI3 kinase then activates protein kinase B (PKB), which mediates SLC2A4 translocation. Insulin resistance develops through diminished PKB and IRS1-associated PI3-kinase activity, as well as by IRS1 degradation. This causes down-regulation of the action of insulin, thereby diminishing the effects of physiological insulin concentrations. Short-term hyperosmotic challenge of adipocytes was shown to inhibit the action of IRS1 through serine phosphorylation. Furthermore, long-term osmotic challenge resulted in IRS1 degradation. Together, these results shed light on the mechanisms underlying hyperosmolarity-induced insulin resistance and substantiate a physiological role of hyperosmotic stress during the initiation and progression of diabetes.

Diabetic nephropathy is the predominant cause of kidney failure in the United States. Hyperosmotic stress is believed to enhance cellular susceptibility to renal tubular fibrosis. This is relevant because tubular fibrosis contributes to the pathogenesis of diabetic nephropathy, which in turn is responsible for >40% of cases that progress to end stage renal disease. Increases in NFAT5 DNA binding activity have been observed in mesangial cells of diabetic patients with diabetic nephropathy, when compared with diabetic control and non-diabetic groups. NFAT5 activity also was elevated in the peripheral blood mononuclear cells of these patients, implying that NFAT5 activation extends beyond cells of the kidney. Glucose-associated increases in plasma solute concentrations are thought to increase hyperosmotic stress within the renal medulla. Renal distal tubule cells exposed to this environment exhibit down-regulation of Smad7. This promotes Smad pathway activation, one effect of which is increased TGFβ receptor stability. The resultant increase in TGFβ signaling is thought to enhance susceptibility of renal tubule cells to extracellular matrix synthesis and fibrosis. The mechanisms underlying injury in diabetic nephropathy parallel those driving diabetic retinopathy, including microvasculature damage associated with hyperosmotic stress. As with other diabetic-related pathologies, it appears that both hyperglycemia-induced osmotic stress and glucose metabolism-associated oxidative stress contribute to the pathogenesis of this condition.

### C. Inflammatory Bowel Disease

Inflammatory bowel disease (IBD) describes a number of inflammatory diseases that affect the gastrointestinal tract. It is characterized by chronic inflammation of one or more regions of the gastrointestinal tract with reoccurring flare-ups. Crohn's disease and ulcerative colitis are the most common forms of IBD. These conditions share many clinical features but differ in location and the nature of the inflammatory changes. Crohn's disease manifests as transmural lesions found anywhere along the gastrointestinal tract and involves multiple cell layers and types. Ulcerative colitis is limited to the epithelial lining of the colon and rectum.

A commonly used mouse model of IBD uses oral administration of dextran sulfate sodium (DSS). A study revealed that DSS feeding causes hyperosmolarity and hyperosmotic stress within the colon, which in turn triggers inflammation (Neuhofer W. Role of NFAT5 in inflammatory disorders associated with osmotic stress. Curr Genomics. 2010; 11:584-590). Other studies in rats using alanine, mannitol, or NaCl as hyperosmotic stimuli produced similar results, suggesting any compound elevating colonic hypertonicity may have pathological consequences. These results corroborate observations in human subjects with IBD, including neonatal necrotizing enterocolitis, Crohn's disease, and ulcerative colitis, where elevated osmolarity in fecal fluid within the colon is noted. Furthermore, the fecal osmolarity elevation in Crohn's disease patients shows a close correlation with disease severity. Under physiological conditions, both pro- and anti-inflammatory cytokines are present within the gastrointestinal tract, with the pro-inflammatory cytokines being kept in check by the anti-inflammatory cytokines. Dysregulation between the pro- and anti-inflammatory signals is believed to be a major factor contributing to the pathogenesis of IBD. Epithelial cells and infiltrating immune cells are believed to be the predominant cytokine-secreting cells in IBD. Cultured intestinal epithelial cells exposed to hyperosmotic media exhibit elevated IL1ß and IL8 production. Studies in human intestinal cells also have revealed NFAT5 activation after treatment with hyperosmotic media. These data are supported by clinical studies documenting IBD patients to have elevated levels of pro-inflammatory cytokines, including TNF, IL1, IL6, and IL18. The overall significance of hyperosmotic stress and epithelial cell cytokine release on IBDs has yet to be determined but represents a potentially important therapeutic target for the treatment of these disorders.

### D. Cardiovascular Disease

Cardiovascular diseases are the leading cause of premature deaths worldwide, being responsible for >17 million deaths in 2008 alone. Hypertension is a major risk factor for coronary heart disease. Osmoreceptor activation in the central nervous system can elevate blood pressure through a mechanism involving increased sympathetic nerve activity, a process thought to contribute to salt-induced hypertension. In rats, a high salt diet activates NFAT5 in macrophages and results in VEGFC secretion. VEGFC then diminishes interstitial hypertonic volume retention by inducing hyperplasia of the lymph capillaries and expression of endothelial nitric oxide synthase. In isolated vascular smooth muscle cells, NFAT5 was shown to be activated by hypertonicity, the vasoconstrictor angiotensin II, and the mitogen platelet-derived growth factor-BB. These effects were selective in that NFAT5 was unaffected by other vasoconstrictors, mitogens, or a variety of cytokines, including IL1B, IL8, IL10, TNF, or IFN. NFAT5 is also significantly up-regulated in models of vascular injury, such as atherosclerotic lesions and neointimal hyperplasia. It was proposed that NFAT5 was involved in the regulation of the vascular smooth muscle cell phenotype. NFAT5 also appears to have a role in the heart. In cardiac myocytes, NFAT5 degradation was found to be a key event mediating doxorubicin cytotoxicity. Exposure of the myocytes to hypertonic media causes NF-κB and caspase activation through a mechanism involving ROS. This would suggest that the hyperosmotic stress triggers oxidative stress and ROS production within the cells. Hyperosmotic stress also triggers apoptosis in cardiac myocytes through a p53-dependent manner. In this respect, it appears to be a more potent stimulus than other factors known to induce cardiac myocyte death, including doxorubicin or angiotensin II. NFAT5 mRNA and protein is up-regulated in cardiac myocytes exposed to hyperosmotic media in vitro. After hyperosmotic challenge with sorbitol, cardiomyocytes also exhibited an increase in AR expression that was accompanied by AR-mediated activation of apoptotic signaling pathways.

The osmolyte taurine is the most abundant free amino acid in cardiac tissues and in skeletal muscle. In response to hyperosmotic stress, cells take up taurine to counter the elevations in extracellular osmolarity. One mechanism responsible for the intracellular taurine accumulation is the NFAT5-mediated up-regulation of the taurine transporter SLC6A6. Consistent with this observation is the finding that SLC6A6 has a NFAT5 binding site within its promoter. Several studies have shown that taurine deficiency leads to cardiomyopathy, suggesting an important role for this osmolyte in maintaining heart health. Due to the multifaceted functions of taurine (including antioxidant and osmoprotectant properties), the precise mechanism(s) mediating protection of cardiac tissues remains to be determined. Using SLC6A6 knockout mice, current evidence suggests that the cytoprotective actions of taurine in cardiac and skeletal muscle are dependent on its ability to act as an osmolyte. These mice exhibited decreased cell volume, a condition often associated with the cell's inability to effectively counter extracellular osmolarity. In addition, SLC6A6 knockout animals exhibited a significant up-regulation of Hsp70, ATA2, and S100A4 within cardiac and skeletal muscle tissues, all of which are induced in response to osmotic stress.

### E. Liver Disease

Osmotic stress can play a role in the pathogenesis of a number of liver disorders. Hepatic hydration heavily influences protein turnover within the liver. A number of factors can cause shrinkage of hepatocytes, including high urea concentrations, amino acid starvation, elevated sodium levels in the blood (hypernatremia), and oxidative stress. Cell shrinkage caused by hepatocellular dehydration leads to an overall increase in proteolytic activity. On the other hand, hepatic protein synthesis is, in turn, reduced under hyperosmotic conditions. The influence of cell shrinkage on protein synthesis has also been observed in mammary tissue. Cellular dehydration is believed to be a driving force behind the severe protein wasting observed within the liver and skeletal muscles of extremely ill patients. The osmolarity of mouse liver is approximately 35 mOsm/kg greater than serum, suggesting this organ is physiologically functioning under slightly hypertonic conditions. In addition, the osmolarity of portal vein blood increases after feeding or ingestion of fluids with a high solute concentration, which subsequently influences hepatic hydration, further underscoring the need for a general response mechanism to hyperosmolar conditions within this tissue. Hydration state greatly influences many hepatic processes. Bile acid uptake and canalicular secretion are both heavily dependent on osmoregulation through transporter integration into the membrane. Hydrophobic bile acids are known to cause hepatocyte shrinkage and it has been speculated that hyperosmotic stress contributes to cholestatic liver injury.

Hyperosmotic stress also sensitizes hepatocytes to apoptosis, suggesting that elevated hepatic osmolarity could influence drug-induced liver injury. The hepatic Na⁺/K⁺/2Cl⁻ co-transporter SLC12A2 (also known as NKCC1) and betaine transporter SLC6A12 (also known as BGT-1) are up-regulated in response to hyperosmotic stress. Both of these proteins are also up-regulated during the transition of hepatic stellate cells to a myofibroblast-like phenotype, suggesting that an intracellular osmolyte imbalance may accompany stellate cell transformation during liver fibrosis. CYP2E1 is up-regulated in hepatocytes exposed to hyperosmotic stress through a NFAT5-dependent mechanism. Such expression increases free radical generation and oxidative stress, which contributes to liver inflammation and fibrosis. CYP2E1 has been linked to a number of liver diseases, including alcoholic liver disease, non-alcoholic steatohepatitis, and cirrhosis. As mentioned above, the amino acid taurine is a physiologically important osmolyte. Hyperosmotic-challenged hepatocytes exhibit up-regulation of the taurine transporter SLC6A6. Knockout mice lacking SLC6A6 protein exhibit severe taurine deficiency and develop chronic liver disease characterized by moderate hepatitis and liver fibrosis. These mice are also more sensitive to ultraviolet B irradiation-induced immunosuppression, suggesting taurine uptake may serve as a protective response under these conditions.

### Disorders and Conditions of the Skin, Mucous Membranes and Hair

Skin, mucous membranes and hair are often exposed to factors, both of a chemical and physical nature, which result in osmotic stress. Osmotic stress can compromise the integrity of the cells, including dehydration of the cells and denaturation or changes in the conformation of proteins (e.g., misfolding of the proteins) in the cell that can exposure hydrophobic residues at the protein surfaces, leading to their susceptibility to forming aggregates and losing their functionality. Osmotic stress further compromises the integrity of the cell by interfering with the DNA damage response; thus, solar radiation, the exposure to certain chemical agents or high temperatures can have harmful effects on the cells which make up the skin, accelerating its aging and making it look unhealthy. The mechanisms through which ultraviolet radiation (UV) exercises these effects includes the formation of reactive oxygen species, damage to the DNA, and the denaturation of proteins, among others.

The HSP polypeptides, compositions and combinations provided herein can be used for cosmetic and/or pharmaceutical treatment (including prevention or reduction) of a variety of conditions associated with the skin, mucous membranes and hair including, but not limited to, burns, sunburns, abrasions, cuts, pimples, allergic reactions, poisoning, exposure to radiation, chemotherapy, contact of the skin with a substance, mechanical friction, dehydration, bed sores, the application of a tattoo and piercing the skin, for slowing down the aging of the skin and/or for rejuvenating/hydrating the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders and children's diseases affecting the skin (such as varicella, rubella, measles), reducing the number and/or the depth of wrinkles, smoothening the skin, restoring the elasticity of the skin, reducing the intensity of age spots, reducing the size of age spots, reducing the number of age spots, reducing hyperpigmentation, to counter dehydration, for improving skin tone/elasticity, skin disorders such as epidermolysis bulosa (a disorder characterized by misfolding of proteins that causes blistering of the skin), for preventing other osmotic stress-induced damage to the skin, such as DNA damage, and the like, or for administration or application to the scalp, such as for reducing hair loss or promoting hair growth in alopecia, including alopecia caused as a side effect of chemotherapy.

### Methods

Certain aspects of the methods provided herein are now described. Provided herein are methods of preventing, reducing or eliminating osmotic stress in a subject, or in one or more cells of a subject, or in one or more cultured cells in vitro, which include, administering to the subject, or to one or more cells of a subject, or to one or more cultured cells in vitro, an amount of at least one HSP polypeptide, or composition or combination thereof, as provided herein, that is sufficient to prevent, reduce or eliminate osmotic stress in the subject, or one or more cells of the subject. In certain aspects, the subject has an inflammatory disorder or condition. In aspects, the disorder or condition is selected from among inflammatory bowel disease, hypernatremia, dry eye syndrome, tissue burns, dehydration, heat stroke, diabetes mellitus, diabetes insipidus, uremia, kidney disease, cardiovascular disease and liver disease. In some aspects, the disease or condition is accompanied by or associated with tissue, including, for example, epithelial tissue, connective tissue, or tissue membranes (e.g., mucous membranes, cutaneous membranes and serous membranes). In certain aspects, the disease or condition can be associated with mucosa, a wound, sore, ulcer or infection. In some aspects, the disorder or condition is associated with the skin, mucous membranes or hair. In aspects, the disorder or condition associated with the skin, mucous membranes or hair is selected from among aging of the skin, dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders and children's diseases affecting the skin (such as varicella, rubella, measles), wrinkles in the skin, rough skin, loss of elasticity in the skin, intense, large and/or numerous age spots, hyperpigmentation, epidermolysis bulosa and alopecia, including alopecia caused as a side effect of chemotherapy.

Also provided herein are methods of treating a disease or condition in a subject that includes administering, to a subject in need thereof, a composition, e.g., pharmaceutical composition or a cosmetic composition for preventing, ameliorating or treating a disease, disorder or condition as provided herein. In some aspects, the methods include administering a therapeutically effective or cosmetically effective amount of a composition, e.g., pharmaceutical or cosmetic composition, or combination as provided herein, for treating a disease or condition as provided herein. In some aspects, the disease, disorder or condition is selected from among inflammatory bowel disease, hypernatremia, dry eye syndrome, tissue burns, dehydration, heat stroke, diabetes mellitus, diabetes insipidus, uremia, kidney disease, cardiovascular disease and liver disease. In aspects, the disease, disorder or condition is one that is susceptible to, associated with and/or accompanied by an infection, tissue, such as animal (e.g., human) tissue, and/or tissue (e.g., surface tissue) injury or damage, including, for example, wounds, sores or burns in or on a subject. In some aspects, the disease or condition is accompanied by or associated with tissue, including, for example, epithelial tissue, connective tissue, or tissue membranes (e.g., mucous membranes, cutaneous membranes and serous membranes). In certain aspects, the disease or condition can be associated with mucosa, a wound, sore, ulcer or infection. In aspects, the disorder or condition is selected from among aging of the skin, dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders and children's diseases affecting the skin (such as varicella, rubella, measles), wrinkles in the skin, rough skin, loss of elasticity in the skin, intense, large and/or numerous age spots, hyperpigmentation, epidermolysis bulosa and alopecia, including alopecia caused as a side effect of chemotherapy.

### Examples of Amino Acid and Nucleotide Sequences

| SEQ ID | | |
|---|---|---|
| NO: | AMINO ACID OR NUCLEOTIDE SEQUENCE | |
| 1 | MAPAVGIDLGTTYSCVGVFREDRCEIIANDQGNRTTPSFVAFTDTE | HSP amino acid |
| | RLIGDAAKNQVAMNPHNTVFDAKRLIGRRENDPEVQSDMKHFPFKV | sequence |
| | VDRNGKPAIEVEFKGEKKVFTPEEISSMVLTKMRETAESYLGTTVT | |
| | NAVITVPAYFNDSQRQATKDAGLIAGLNVLRIINEPTAAAIAYGLD | |
| | KKADGERNVLIFDLGGGTFDVSLLTIEEGIFEVKSTAGDTHLGGED | |
| | FDNRLVNHLCQEFKRKFKKDLTTNARALRRLRTAAERAKRTLSSAA | |
| | QTSIEIDSLFEGIDFYTSITRARFEELCQDLFRSTLQPVDRVLTDA | |
| | KIDKSQVHEIVLVGGSTRIPRIQKLITDYFNGKEPNKSINPDEAVA | |
| | YGAAVQAAILSGDTTSKSTNEILLLDVAPLSLGIETAGGIMTKLIP | |
| | RNTTIPTKKSEVFSTFSDNQPGVLIQVYEGERQRTKDNNLLGKFEL | |
| | TGIPPAPRGVPQIEVTFDLDANGIMNVSALEKGTGKTNQIVITNDK | |
| | GRLSKEEIERMLAEAEKFKEEDEREARRVQAKNGLESYAYSLRNTL | |
| | NDPKVDEKLDASDKEKLRSEIEKIVNWLDENQQASREEYEDQQKEL | |
| | ESIANPIMMKFYGAGAGGMPGGMPGGAPGGFPSAGAGSHDNEGPTV | |
| | EEVD | |
| 2 | HHHHHHSSGLVPRGSHMAPAVGIDLGTTYSCVGVFREDRCEIIAND | recombinantly |
| | QGNRTTPSFVAFTDTERLIGDAAKNQVAMNPHNTVFDAKRLIGRRF | expressed HSP with |
| | NDPEVQSDMKHFPFKVVDRNGKPAIEVEFKGEKKVFTPEEISSMVL | N-terminus His tag |
| | TKMRETAESYLGTTVTNAVITVPAYENDSQRQATKDAGLIAGLNVL | and thrombin |
| | RIINEPTAAAIAYGLDKKADGERNVLIFDLGGGTFDVSLLTIEEGI | cleavage sites for |
| | FEVKSTAGDTHLGGEDFDNRLVNHLCQEFKRKFKKDLTTNARALRR | purification |
| | LRTAAERAKRTLSSAAQTSIEIDSLFEGIDFYTSITRARFEELCOD | |
| | LFRSTLQPVDRVLTDAKIDKSQVHEIVLVGGSTRIPRIQKLITDYF | |
| | NGKEPNKSINPDEAVAYGAAVQAAILSGDTESKSTNEILLLDVAPL | |
| | SIGIETAGGIMTKLIPRNTTIPTKKSEVFSTFSDNQPGVLIQVYEG | |
| | ERQRTKDNNLLGKFELTGIPPAPRGVPQIEVTFDLDANGIMNVSAL | |
| | EKGTGKTNQIVITNDKGRLSKEEIERMLAEAEKFKEEDEREARRVQ | |
| | AKNGLESYAYSLRNTLNDPKVDEKLDASDKEKLRSEIEKIVNWLDE | |
| | NQQASREEYEDQQKELESIANPIMMKFYGAGAGGMPGGMPGGAPGG | |
| | FPSAGAGSHDNEGPTVEEVD | |
| 3 | ATGGCTCCCGCCGTCGGCATTGACTTGGGCACGACCTACTCGTGCG | nucleic acid |
| | TGGGCGTGTTCCGCGAGGATCGCTGCGAAATCATCGCCAATGACCA | sequence derived |
| | GGGCAACCGGACTACTCCGTCGTTCGTGGCCTTCACGGACACGGAG | from HSP of SEQ ID |
| | CGTCTGATCGGTGATGCCGCCAAGAACCAGGTCGCCATGAACCCTC | NO: 1 |
| | ACAACACCGTCTTCGACGCCAAGCGCCTCATCGGCCGCCGGTTCAA | |
| | CGACCCCGAGGTCCAGTCCGACATGAAGCACTTCCCGTTCAAGGTC | |
| | GTTGATCGGAACGGCAAGCCGGCCATCGAGGTCGAGTTCAAGGGCG | |
| | AGAAGAAGGTCTTCACTCCCGAGGAGATCTCGTCCATGGTCCTGAC | |
| | CAAGATGCGCGAGACGGCCGAGTCGTACCTGGGCACCACCGTCACC | |
| | AACGCCGTCATCACCGTCCCTGCCTACTTCAACGACTCCCAACGCC | |
| | AGGCCACCAAGGACGCTGGTCTGATTGCCGGCCTCAACGTCCTGCG | |
| | TATCATCAACGAGCCCACCGCCGCTGCCATTGCCTACGGTCTCGAC | |
| | AAGAAGGCCGATGGTGAGCGCAACGTCCTGATCTTCGACCTCGGTG | |
| | GTGGCACCTTCGATGTCTCGCTCCTCACCATCGAGGAAGGCATCTT | |
| | CGAGGTCAAGTCCACCGCTGGTGACACCCACCTGGGTGGTGAGGAC | |
| | TTCGACAACCGGCTCGTGAACCATTTATGCCAAGAATTTAAGCGCA | |
| | AGTTCAAGAAGGACCTCACGACCAATGCACGCGCCCTGCGCCGCCT | |
| | GCGCACCGCTGCCGAGCGTGCCAAGCGCACTCTATCGAGCGCTGCC | |
| | CAGACCAGCATCGAGATCGACTCGCTCTTCGAGGGTATTGACTTCT | |
| | ACACGTCGATCACCCGTGCTCGTTTCGAGGAGCTCTGCCAGGACCT | |
| | GTTCCGTTCCACCCTCCAGCCGGTGGACCGCGTCCTCACGGATGCC | |
| | AAGATCGACAAGTCGCAGGTCCATGAGATCGTGCTCGTCGGCGGCT | |
| | CGACCCGTATCCCGCGCATCCAGAAGCTCASCACCGACTACTTCAA | |
| | CGGCAAGGAGCCCAACAAGTCCATCAACCCCGACGAGGCCGTTGCC | |
| | TACGGCGCCGCCGTTCAGGCCGCCATCCTGTCGGGCGACACGACTT | |
| | CTAAGTCGACCAACGAGATCCTGCTTCTCGATGTCGCTCCTCTGTC | |
| | GCTCGGCATCGAGACCGCTGGCGGCATCATGACCAAGCTCATCCCG | |
| | CGCAACACGACCATCCCGACCAAGAAGTCCGAGGTGTTCTCGACCT | |
| | TCTCCGACAACCAGCCCGGTGTGCTCATCCAGGTCTACGAGGGTGA | |
| | GCGCCAGCGCACCAAGGACAACAACCTGCTCGGCAAGTTCGAGCTT | |
| | ACCGGCATTCCGCCGGCTCCTCGCGGTGTGCCCCAGATCGAGGTCA | |
| | CATTCGACCTGGATGCCAACGGCATCATGAACGTGTCGGCCCTCGA | |
| | GAAGGGCACGGGCAAGACGAACCAGATCGTCATTACCAATGACAAG | |
| | GGCCGCCTGTCCAAGGAGGAGATTGAGCGCATGCTTGCCGAGGCCG | |
| | AGAAGTTCAAGGAGGAGGACGAGCGCGAGGCTCGCCGTGTCCAGGC | |
| | CAAGAACGGCCTTGAGTCGTACGCCTACTCGCTGCGCAACACGCTC | |
| | AACGACCCCAAGGTGGACGAGAAGCTCGACGCCTCCGACAAGGAGA | |
| | AGCTGCGCTCCGAGATCGAAAAGATTGTCAACTGGCTCGACGAGAA | |
| | CCAGCAGGCCAGCCGCGAGGAGTACGAGGACCAACAGAAGGAGCTC | |
| | GAGTCCATTGCCAACCCGATCATGATGAAGTTCTATGGTGCTGGCG | |
| | CCGGTGGCATGCCCGGTGGCATGCCCGGTGGCGCCCCTGGTGGCTT | |
| | CCCCAGCGCCGGTGCCGGCAGCCACGACAACGAGGGCCCGACTGTC | |
| | GAGGAGGTCGACTAA | |
| 4 | CATCATCATCATCATCACAGCAGCGGCCTGGTGCCGCGCGGCAGCC | nucleic acid |
| | ATATGGCTCCCGCCGTCGGCATTGACTTGGGCACGACCTACTCGTG | sequence derived to |
| | CGTGGGCGTGTTCCGCGAGGATCGCTGCGAAATCATCGCCAATGAC | recombinantly |
| | CAGGGCAACCGGACTACTCCGTCGTTCGTGGCCTTCACGGACACGG | express HSP of |
| | AGCGTCTGATCGGTGATGCCGCCAAGAACCAGGTCGCCATGAACCC | SEQ ID NO: 2 |
| | TCACAACACCGTCTTCGACGCCAAGCGCCTCATCGGCCGCCGGTTC | |
| | AACGACCCCGAGGTCCAGTCCGACATGAAGCACTTCCCGTTCAAGG | |
| | TCGTTGATCGGAACGGCAAGCCGGCCATCGAGGTCGAGTTCAAGGG | |
| | CGAGAAGAAGGTCTTCACTCCCGAGGAGATCTCGTCCATGGTCCTG | |
| | ACCAAGATGCGCGAGACGGCCGAGTCGTACCTGGGCACCACCGTCA | |
| | CCAACGCCGTCATCACCGTCCCTGCCTACTTCAACGACTCCCAACG | |
| | CCAGGCCACCAAGGACGCTGGTCTGATTGCCGGCCTCAACGTCCTG | |
| | CGTATCATCAACGAGCCCACCGCCGCTGCCATTGCCTACGGTCTCG | |
| | ACAAGAAGGCCGATGGTGAGCGCAACGTCCEGATCTTCGACCTCGG | |
| | TGGTGGCACCTTCGATGTCTCGCTCCTCACCATCGAGGAAGGCATC | |
| | TTCGAGGTCAAGTCCACCGCTGGTGACACCCACCTGGGTGGTGAGG | |
| | ACTTCGACAACCGGCTCGTGAACCATTTATGCCAAGAATTTAAGCG | |
| | CAAGTTCAAGAAGGACCTCACGACCAATGCACGCGCCCTGCGCCGC | |
| | CTGCGCACCGCTGCCGAGCGTGCCAAGCGCACTCTATCGAGCGCTG | |
| | CCCAGACCAGCATCGAGATCGACTCGCTCTTCGAGGGTATTGACTT | |
| | CTACACGTCGATCACCCGTGCTCGTTTCGAGGAGCTCTGCCAGGAC | |
| | CTGTTCCGTTCCACCCTCCAGCCGGTGGACCGCGTCCTCACGGATG | |
| | CCAAGATCGACAAGTCGCAGGTCCATGAGATCGTGCTCGTCGGCGG | |
| | CTCGACCCGTATCCCGCGCATCCAGAAGCTCATCACCGACTACTTC | |
| | AACGGCAAGGAGCCCAACAAGTCCATCAACCCCGACGAGGCCGTTG | |
| | CCTACGGCGCCGCCGTTCAGGCCGCCATCCEGTCGGGCGACACGAC | |
| | TTCTAAGTCGACCAACGAGATCCTGCTTCTCGATGTCGCTCCTCTG | |
| | TCGCTCGGCATCGAGACCGCTGGCGGCATCATGACCAAGCTCATCC | |
| | CGCGCAACACGACCATCCCGACCAAGAAGTCCGAGGTGTTCTCGAC | |
| | CTTCTCCGACAACCAGCCCGGTGTGCTCATCCAGGTCTACGAGGGT | |
| | GAGCGCCAGCGCACCAAGGACAACAACCTGCTCGGCAAGTTCGAGC | |
| | TTACCGGCATTCCGCCGGCTCCTCGCGGTGTGCCCCAGATCGAGGT | |
| | CACATTCGACCTGGATGCCAACGGCATCATGAACGTGTCGGCCCTC | |
| | GAGAAGGGCACGGGCAAGACGAACCAGATCGTCATTACCAATGACA | |
| | AGGGCCGCCTGTCCAAGGAGGAGATTGAGCGCATGCTTGCCGAGGC | |
| | CGAGAAGTTCAAGGAGGAGGACGAGCGCGAGGCTCGCCGTGTCCAG | |
| | GCCAAGAACGGCCTTGAGTCGTACGCCTACECGCTGCGCAACACGC | |
| | TCAACGACCCCAAGGTGGACGAGAAGCTCGACGCCTCCGACAAGGA | |
| | GAAGCTGCGCTCCGAGATCGAAAAGATTGTCAACTGGCTCGACGAG | |
| | AACCAGCAGGCCAGCCGCGAGGAGTACGAGGACCAACAGAAGGAGC | |
| | TCGAGTCCATTGCCAACCCGATCATGATGAAGTTCTATGGTGCTGG | |
| | CGCCGGTGGCATGCCCGGTGGCATGCCCGGEGGCGCCCCTGGTGGC | |
| | TTCCCCAGCGCCGGTGCCGGCAGCCACGACAACGAGGGCCCGACTG | |
| | TCGAGGAGGTCGACTAA | |

### EXAMPLES

The examples set forth below illustrate certain embodiments and do not limit the technology. Certain examples set forth below utilize standard recombinant DNA, membrane vesicle/liposome preparation and other biotechnology protocols known in the art.

### Example 1: Expression and Purification of HSP

DNA (SEQ ID NO:4), which is a nucleotide sequence derived from an experimentally obtained amino acid sequence encoding the HSP as modified to have the sequence set forth in SEQ ID NO:2, was introduced into a plasmid for recombinant expression of the HSP, followed by purification of the recombinant expressed HSP, as follows:

### A. Bacterial Transformation Protocol

Chemically competent Rosetta™ DE3 pLysS cells (Millipore) were transformed by adding 5 μl of 10 ng/μl (50 ng total) of pET28b (+)-Hsp70 plasmid DNA (Integrated DNA Technologies) to a 50 μl aliquot of cells. The plasmid-cell mixture was incubated on ice for 20 mins, then heated to 42° C. for 90 secs. on a benchtop heat block. The cells were transferred to ice and incubated for 2 mins. A total of 445 μl of sterile SOC media (provided by Millipore with competent Rosetta™ cells) was added to the transformation mixture and incubated for 1 hour on a benchtop 37° C. incubator shaking at 225 rpm. After recovery, the cells were centrifuged at 6,000 rpm for 5 mins on a benchtop microfuge. A portion of the supernatant (300 μl) was removed and discarded into a waste container. The cell pellet was resuspended in the remaining 200 μl and delivered to the center of a LB-agar plate containing 30 μg/ml kanamycin and 34 μg/ml chloramphenicol via pipette. The solution was spread across the entire surface of the plate using a freshly ethanol treated and flamed glass cell spreader. The plate was inverted and incubated at 37° C. overnight (O/N), ˜16 hrs.

### B. Scale-Up, Growth and Induction

A total of 3 colonies were picked from the transformation LB-agar plate and deposited into 200 ml of autoclaved LB broth containing kanamycin and chloramphenicol at the concentrations listed in part A. above (added post-autoclaving). The culture was grown O/N (overnight; ˜ 16 hrs.) at 37° C. constant shaking at 225 rpm. A 25 ml portion of the O/N culture was transferred to 500 ml of LB and antibiotics in a 1 L Erlenmeyer flask. A total of 4 flasks were back diluted, resulting in a total of 2 L of culture. The culture was grown to and OD₆₀₀ between 0.6 and 0.8 (˜2-3 hrs.). A 500 μl volume of 1 M IPTG was added to each flask (final concentration 1 mM). The cultures were induced O/N at room temperature on an orbital shaker set to 225 rpm. The cells were transferred to 500 ml centrifuge bottles and pelleted at 6,000×g for 20 mins. The supernatant was discarded, and the cell pellets were frozen at −80° C. until needed for further processing.

### C. Cell Lysis

The cell pellets were removed from the centrifuge bottles using a clean, ethanol-treated spatula. The pellets were placed in a 200 ml glass beaker with 30 ml of Binding Buffer: 50 mM sodium phosphate 7.4, 500 mM sodium chloride, 40 mM imidazole, supplemented with: 150 μl 10 mg/ml or 10 U/ml DNase I from bovine pancreas, 300 μl of 25 mg/ml lysozyme, 3-EDTA-free Pierce Protease Inhibitor Cocktail (tablets), 1 mM TCEP and 1% Triton X-100, and mixed using a magnetic stir bar on a magnetic stir plate housed in a 4° C. cold-cabinet for ˜1 hr. When the solution was visibly homogeneous, the stir bar was removed. The cell suspension was transferred to a 50 ml conical tube and lysed using a 5-50 ml sonicator probe with the following settings: amplitude 80%, 30 sec. on/30 sec. off for a total lysis time of 15 mins. The cell lysate was then centrifuged at 29.097×g for 30 mins at 4° C. The lysate (supernatant) was filtered through a 0.2 μm PES syringe filter into a clean 200 ml plastic graduated cylinder.

### D. Purification of His-Tagged HSP Via FPLC Driven IMAC and Confirmed Using SDS-PAGE Analysis

The ˜30 ml of cell lysate was loaded over a 20 ml HiPrep column packed with Sepharose 6 IMAC resin (Cytiva) charged with nickel sulfate at 5 ml/min using the A line of an AKTA Explorer 100 FPLC. Line A and the attached HiPrep column were pre-equilbrated in 5 column volumes (CV) of Binding Buffer (50 mM sodium phosphate pH 7.4, 500 mM sodium chloride, 40 mM imidazole) at 5 ml/min. The HiPrep column was washed with Binding Buffer until the A280 trace returned to baseline (˜8 CV). Elution buffer (50 mM sodium phosphate pH: 7.4, 500 mM sodium chloride, 500 mM imidazole) was used to equilibrate line B of the AKTA FPLC. Protein bound to the HiPrep column was eluted using the following method: Flowrate: 5 ml/min, Gradient: 0% B-46% B in 200 ml, collected 5 ml fractions. Fractions that fell under the A280 peak in the chromatogram were sampled for SDS-PAGE (30 μl+10 μl SDS-sample buffer) and boiled for 5 minutes at 95° C. The samples were loaded onto a 4-20% Tris-glycine polyacrylamide gel (NuSep). The gel was run at 150 V (constant) for ˜45 mins. until the dye front from the sample buffer exited the bottom of the gel. The gel was stained O/N in AcquaStain (Bulldog Bio).

### E. Alternate Purification of HSP Using Gravity-Flow Column Chromatography

A total of 5 ml of a 50% slurry of HisPur Ni-NTA resin (Thermo) was transferred to a 50 ml conical tube and centrifuged at 700×g for 2 mins. The supernatant was removed using a 25 ml disposable serological pipette. The resin was resuspended in 25 ml of Binding Buffer and mixed by inversion on a rotating end over end mixer for 2 minutes at 4° C. The conical was centrifuged again at 700×g and the supernatant was discarded. The resin was then resuspended in the 30 ml of cell lysate supernatant post centrifugation (see above) and incubated at 4° C. for 1.5 hrs. on an end over end mixer. The Ni-NTA-lysate mixture was poured into a HisPur Ni-NTA Spin Column (Thermo Fisher-88226) and packed by gravity flow. The flowthrough was collected in a glass waste beaker. The column was washed in 5 (25 ml total) CV of Binding Buffer. Bound protein was eluted with Elution Buffer (50 mM sodium phosphate pH 7.4, 300 mM sodium chloride, 250 mM imidazole) in 4-5 ml fractions collected in separate 15 ml conical tubes. Samples from each fraction (30 μl) were taken and processed for SDS-PAGE analysis (as described in part D. above). Samples were analyzed for purity and the purest samples were pooled dialyzed, concentrated, and quantified as described in the section below.

### F. Dialysis, Concentration and Quantification

Fractions that showed the highest purity were pooled and loaded into 12 ml 10 kDa MWCO Slide-a-lyzer Dialysis Cassettes (Fisher) and dialyzed against 50 mM Tris pH: 7.4, 150 mM sodium chloride. The cassettes were dialyzed initially into 4 L for 2-3 hours. The dialysis buffer was changed, and the cassettes were incubated in fresh buffer (4-6 L) O/N. The protein solution was extracted from the cassettes and centrifuged at 29,097×g for 20 minutes. The supernatant was removed and concentrated using Vivaspin 20, 10 kDa MWCO centrifugal concentrators to ˜1 ml volume. Samples (5 μl) were taken from each concentrated aliquot and processed for SDS-PAGE by mixing with 1.7 μl of SDS-sample buffer and boiling as described above. The gels were run and stained as previously described. The protein concentration of pure samples was determined by using the molecular weight (73,035.14 Da), extinction coefficient (23,630 M 1 cm-1) and absorbance at 280 nm on a nanodrop spectrophotometer (Thermo) to calculate a mg/ml concentration using the nanodrop software.

### Example 2: Osmoprotective Activity of HSP

### A. Purpose

This assay procedure was used to screen the recombinantly produced HSP for its ability to protect against osmotic stress, using cultured human keratinocytes as the testing model. The assay used changes in cell viability as the endpoint for measuring the protective effect.

### B. Summary of Test Method

For this study, human keratinocytes were pretreated for 24 hours with either HSP, a mild hypertonic solution of sorbitol (positive control), or left untreated (negative control). After the pretreatment, the cell culture media was replaced with media supplemented with NaCl at progressively increasing hypertonic concentrations. The cells were then incubated again for 24 hours. After the second incubation, cell viability was determined using an MTT assay.

The MTT assay is a colorimetric analysis of the metabolic activity of the cell, which is a reflection of cell viability. Viable cells can take up MTT, which is then reduced by mitochondria, resulting in the formation of insoluble purple formazin crystals. These crystals are then extracted from the cells with isopropanol and quantified spectrophotometrically. The intensity of the purple color is directly proportional to the number of viable cells.

### C. Methods

### Human Keratinocyte Cell Culture

Human adult epidermal keratinocytes were grown using EpiLife Media (60 UM calcium) supplemented with 0.2% v/v bovine pituitary extract, 1 μg/ml recombinant human insulin-like growth factor-1, 0.18 μg/ml hydrocortisone, 5 μg/ml bovine transferrin, 0.2 ng/ml human epidermal growth factor. When a sufficient number of cells had been grown, they were transferred to 96-well plates and cultured for a minimum of 24 hours to allow the cells to adhere to the well plates. The cells were then used when they were fully confluent.

### Pretreatment of Keratinocytes

The test materials and the sorbitol positive controls were prepared in fully supplemented EpiLife media. Supplemented EpiLife media alone served as the untreated control. These solutions were applied to the keratinocytes in the 96-well plates, after which the plates were incubated for 24 hours at 37±2° C. and 5±1% CO2.

### Osmotic Stress

After the pretreatment incubation, the cell culture media was removed from both of the 96-well plates and replaced with supplemented EpiLife media which had progressively increasing concentrations of additional NaCl added (0, 75, 100, 125 and 150 mM) to generate a series of increasingly hypertonic solutions. After the addition of the media/NaCl, the 96-well plates were incubated for an additional 24 hours.

### MTT Assay

After the second 24-hour incubation, the cell culture media was removed and the cells were washed once with phosphate buffered saline. After washing, 100 μl of keratinocyte cell culture media supplemented with MTT (0.5 mg/ml) was added to each well. The 96-well plates were returned to the incubator (37+2° C. and 5+1% CO2) for one hour. After the incubation the MTT solution was removed via aspiration and the cells were rinsed twice with phosphate buffered saline to remove any residual MTT on the outside of the cells and then 100 μl of isopropyl alcohol was added to the wells to extract the purple formazin crystals. The well plates were then read at 540 nm using isopropyl alcohol as a blank.

### D. Calculations

### MTT Assay

The mean MTT absorbance value for the untreated control cells not exposed to any osmotic stress (0 mM NaCl) was calculated and used to represent 100% cell viability. The individual MTT values from the cells undergoing the various treatments were then divided by the mean value for the negative control cells and expressed as a percentage to determine the change in cell viability caused by each treatment.

### E. Results

The results for the MTT assay are presented in Table 1. MTT scores for each treatment are expressed as percent mean cell viability±the standard deviation of the mean. The data shown is percent viability of the cells in solutions of increasing hypertonicity (mM NaCl), when the cells are either untreated or subjected to various treatments as shown:

TABLE 1
Cell Viability

| Treatment | 0 mM NaCl | 75 mM NaCl | 100 mM NaCl | 125 mM NaCl | 150 mM NaCl |
|---|---|---|---|---|---|
| Untreated | 100.0 +/− 14.6 | 92.9 +/− 15.3** | 65.6 +/− 9.6** | 41.5 +/− 3.5** | 12.5 +/− 6.9** |
| 10% HSP | 158.5 +/− 14.1* | 171.5 +/− 11.1* | 140.5 +/− 8.8* | 67.6 +/− 4.3* | 15.5 +/− 2.0 |
| 5% HSP | 157.5 +/− 10.2* | 177.6 +/− 10.8* | 122.9 +/− 9.0* | 70.5 +/− 3.9* | 24.8 +/− 6.0 |
| 1% HSP | 160.7 +/− 18.1* | 177.1 +/− 8.3* | 123.1 +/− 9.0* | 81.6 +/− 7.9* | 24.8 +/− 8.7 |
| 25 mM Sorbitol | 121.6 +/− 9.6 | 100.0 +/− 7.7 | 93.9 +/− 2.8* | 69.5 +/− 9.7* | 29.5 +/− 5.6 |
| 12.5 mM Sorbitol | 100.7 +/− 13.0 | 103.2 +/− 1.8 | 98.3 +/− 6.0* | 73.2 +/− 13.3* | 24.1 +/− 9.6 |

*Denotes cell viabilities which are significantly different from the Untreated group when comparing treatments within the same amount of NaCl added.
**Denotes significant differences in the viability of just the Untreated group when comparing viabilities in the solutions containing different amounts of added NaCl.

### F. Discussion

The purpose of this study was to test the ability of HSP to protect against osmotic stress. For this study, cultured keratinocytes were pretreated with HSP, sorbitol (positive control) or left untreated (negative control) and then osmotic stress was induced by the addition of cell culture media containing various amounts of added NaCl to generate a series of progressively increasing hypertonic solutions. In this study, as the NaCl concentration increased, there was a dose dependent decrease in the viability of the untreated keratinocytes in response to the osmotic stress generated by the increasing hypertonic conditions. With this model, treatments/agents that can prevent or delay this decrease in cell viability in response to increasingly hypertonic conditions are considered to provide protection from osmotic stress.

In this study, both the HSP and the sorbitol positive control were observed to slow the reduction in cell viability caused by the increase in hypertonicity. In the untreated group, cell viability was observed to significantly decrease starting at 100 mM of additional NaCl, with further decreases observed at the 125 mM and 150 mM levels. At both 100 mM and 125 mM NaCl, cell viability in the HSP and sorbitol treated cell samples was significantly greater than the untreated cell samples at these two concentrations. Cell viability in the HSP-treated cell samples was significantly greater than the untreated cell samples at all hypertonicities tested except the sample with the highest concentration of added NaCl (150 mM), where neither HSP nor sorbitol (positive control) showed a significant osmoprotective effect.

The results demonstrate that the HSP polypeptides provided herein can protect against osmotic stress. Interestingly, cell viability in keratinocytes treated with HSP and then exposed to lower concentrations of added NaCl (0 mM and 75 mM), which did not impact cell viability in the untreated group, displayed a significant improvement. This increase in cell viability was not observed in the toxicity prescreen, in which the cells were treated with the material continuously for 48 hours, and may be due to the different treatment regimen in this study where the cells were treated for 24 hours with the material and then grown an additional 24 hours without it.

### Example 3: Antioxidant Activity of HSP

### A. Purpose

This assay procedure was used to screen materials for antioxidant properties using non-cell-based methods. Dilutions of HSP tested were 10%, 5%, 1%, 0.5%, 0.1%, 0.05%, and 0.01%.

### B. Summary of Test Methods

Fluorescein (FITC) is normally a highly fluorescent molecule, yet when fluorescein is oxidized by either peroxyl radicals or hydroxyl radicals, it loses its fluorescence. Thus, when FITC is incubated in the presence of 2,2′-azobis(2-amidino-propane) dihydrochloride (AAPH, a peroxyl radical generating compound), or a combination of hydrogen peroxide and cobalt (a hydroxyl radical generating system based on the Fenton-type reaction), there will be a time dependent loss of FITC fluorescence. However, if a material with antioxidant properties is present during the incubation, the loss of fluorescence will be delayed as some of the peroxyl or hydroxyl radicals react with the antioxidant material instead of the FITC. Under these conditions, the delay or prevention in fluorescence decay will be in proportion to the antioxidant capacity of the test material.

### C. Methods

### HORAC Assay (OxiSelect HORAC Assay, Cell Biolabs)

The HORAC assay was performed as specified by the manufacturer of the kit. For the assay, the test materials were prepared in Assay Buffer at 10× their final desired concentrations. Caffeic acid was used as the positive control for this assay. To start the assay, 20 μl of the test material or positive control were added to the wells of a 96-well plate. All of the samples were prepared in triplicate. Next, 140 μl of an FITC solution was added to each well and the plate was incubated for 30 minutes in the dark. After this incubation, 20 μl of a hydrogen peroxide solution (Hydroxyl Radical Initiator) was added to each well, followed by the addition of 20 μl of a cobalt solution (Fenton Reagent). The plate was mixed and then read using a Fluoroskan Ascent Fluorometer at 3-minute intervals for 60 minutes with an excitation wavelength of 480 nm and an emission wavelength of 518 nm.

### ORAC Assay

For the ORAC assay, the test materials were prepared in Assay Buffer at 10× their final desired concentrations. Trolox was used as the positive control for this assay. To start the assay, 20 μl of the test material or positive control were added to the wells of a 96-well plate. All of the samples were prepared in triplicate. Next, 160 μl of a 360 nM FITC solution was added to each well and the plate was incubated for 30 minutes in the dark. After this incubation, 20 μl of a 153 mM AAPH (peroxyl radical generator) was added to each well. The plate was mixed and then read using a Fluoroskan Ascent Fluorometer at 2.5-minute intervals for 60 minutes with an excitation wavelength of 480 nm and an emission wavelength of 518 nm.

### D. Calculations

The loss of FITC fluorescence can be graphed by plotting fluorescence intensity vs time for each of the samples to generate a fluorescence decay curve. The area under this curve (AUC) then represents the extent of FITC fluorescence loss over the course of the assay, and this measurement is then used as an index for the effectiveness of the antioxidant samples. The AUC for a sample can be calculated using the following equation:

$$AUC = 1 + ( RFU 1 / RFU 0 ) + ( RFU 2 / RFU 0 ) + \text{…} + ( RFU final / RFU 0 )$$

Where RFUO is the initial fluorescence measurement of the sample at time 0, with each subsequent RFU measurement indicated by the respective increased numbers. The AUC measurements were then used to determine the percent inhibition for each concentration of the test material screened, followed by the determination of the IC50, the concentration of the material at which 50% of the FITC signal loss due to radical interaction is prevented.

### E. Results

The results for the HORAC assay and ORAC assay are presented below. HSP was observed to scavenge both hydroxyl radicals (HORAC) and peroxyl radicals (ORAC) at the IC50 levels provided in Table 3 and Table 5.

TABLE 2
Caffeic Acid HORAC

| | Concentration (ug/ml) | | | | | | |
|---|---|---|---|---|---|---|---|
| | 20 | 10 | 5 | 2.5 | 1.25 | 0.625 | 0 |
| Percent | 62.58 | 60.03 | 68.86 | 56.30 | 14.56 | 8.85 | 0 |
| Inhibition | | | | | | | |

IC50: 3.25 ug/ml

TABLE 3
HSP HORAC

| | Concentration Percent (v/v) | | | | | | | |
|---|---|---|---|---|---|---|---|---|
| | 10 | 5 | 1 | 0.5 | 0.1 | 0.05 | 0.01 | 0 |
| Percent | 77.3 | 66.83 | 39.58 | 32.15 | 7.67 | −18.88 | 10.84 | 0 |
| Inhibition | | | | | | | | |

IC50: 2.68%

TABLE 4
Trolox ORAC

| | Concentration (ug/ml) | | | | | | | |
|---|---|---|---|---|---|---|---|---|
| | 5 | 2.5 | 1.25 | 0.625 | 0.3125 | 0.15625 | 0.078125 | 0 |
| Percent | 90.3 | 87.4 | 84.06 | 83.84 | 68.77 | 47.09 | 30.79 | 0 |
| Inhibition | | | | | | | | |

IC50: 0.42 ug/ml

TABLE 5
HSP ORAC

| | Concentration Percent (v/v) | | | | | | | |
|---|---|---|---|---|---|---|---|---|
| | 10 | 5 | 1 | 0.5 | 0.1 | 0.05 | 0.01 | 0.0 |
| Percent | 68.11 | 73.06 | 41.74 | 26.26 | 1.84 | −6.1 | −5.14 | 0.00 |
| Inhibition | | | | | | | | |

IC50: 2.48%

### Non-Limiting Embodiments

Listed hereafter are non-limiting examples of certain embodiments of the technology.

A1. An isolated, recombinant or synthetically produced polypeptide comprising a consecutive sequence of amino acids that is more than 95% identical to a corresponding consecutive sequence of amino acids set forth in SEQ ID NO:1.

A2. The polypeptide of embodiment A1, comprising a consecutive sequence of amino acids that is 96%, 97%, 98%, or 99% or more identical, or 100% identical, to a corresponding consecutive sequence of amino acids set forth in SEQ ID NO: 1.

A3. An isolated, recombinant or synthetically produced polypeptide comprising a consecutive sequence of amino acids that is more than 95% identical to a corresponding consecutive sequence of amino acids set forth in SEQ ID NO:2.

A4. The polypeptide of embodiment A3, comprising a consecutive sequence of amino acids that is 96%, 97%, 98%, or 99% or more identical, or 100% identical, to a corresponding consecutive sequence of amino acids set forth in SEQ ID NO: 2.

A5. A polypeptide of any one of embodiments A1-A4, wherein the polypeptide or a portion thereof exhibits osmoprotective activity.

A6. The polypeptide of embodiment A5, wherein the osmoprotective activity comprises preventing hyperosmotic stress.

A7. The polypeptide of embodiment A5 or A6, wherein the osmoprotective activity comprises reducing or eliminating hyperosmotic stress.

A8. The polypeptide of any one of embodiments A1-A7, for use in the prevention, reduction or elimination of a disease, disorder or condition associated with inflammation, or with a disease, disorder or condition of the skin, mucosal membranes or hair, or for use as an antioxidant.

A9. The polypeptide of embodiment A8, wherein the disease, disorder or condition is selected from among inflammatory bowel disease, hypernatremia, dry eye syndrome, tissue burns, dehydration, heat stroke, diabetes mellitus, diabetes insipidus, uremia, kidney disease, cardiovascular disease and liver disease.

A10. The polypeptide of embodiment A9, wherein the disease, disorder or condition is one that is susceptible to infection, injury or damage in or on a subject.

A11. The polypeptide of embodiment A9 or A10, wherein the disease or condition is associated with mucosa, a wound, sore, ulcer or infection.

A12. The polypeptide of any one of embodiments A8-A11, wherein the disease, disorder or condition is selected from among burns, sunburns, abrasions, cuts, pimples, allergic reactions, poisoning, exposure to radiation, chemotherapy, contact of the skin with a substance, mechanical friction, dehydration, bed sores, the application of a tattoo and piercing the skin, aging of the skin and/or dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, i aging of the skin, dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders and children's diseases affecting the skin (such as varicella, rubella, measles), wrinkles in the skin, rough skin, loss of elasticity in the skin, intense, large and/or numerous age spots, hyperpigmentation, epidermolysis bulosa and alopecia, including alopecia caused as a side effect of chemotherapy.

A13. The polypeptide of any one of embodiments A1-A12, wherein the amount of the one or more polypeptides is effective to increase the viability of one or more cells subjected to hyperosmotic stress by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%, 185%, 190%, 195%, 200%, 300%, 400% or more, relative to the viability of the one or more cell under hyperosmotic stress that is/are not subjected to treatment with the polypeptide.

B1. An isolated or recombinant polynucleotide encoding the polypeptide of any one of embodiments A1-A13.

B2. The polynucleotide of embodiment B1, comprising a consecutive sequence of nucleotides that is 90% or more identical to a corresponding consecutive sequence of nucleotides set forth in SEQ ID NO: 3 or SEQ ID NO: 4.

B3. The polynucleotide of embodiment B2, comprising a consecutive sequence of nucleotides that is 90%, 91%, 92%, 93%, 94%, 95%, 96%, 97%, 98%, 99% or 100% identical to a corresponding consecutive sequence of nucleotides set forth in SEQ ID NO:3 or SEQ ID NO:4.

B4. The polynucleotide of any one of embodiments B1-B3, wherein the polypeptide encoded by the polynucleotide exhibits osmoprotective activity.

B5. The polynucleotide of embodiment B4, wherein the osmoprotective activity comprises preventing hyperosmotic stress.

B6. The polynucleotide of embodiment B4 or B5, wherein the osmoprotective activity comprises reducing or eliminating hyperosmotic stress.

C1. The polypeptide of any one of embodiments A1-A13, which is thermally stable.

C2. The polynucleotide of any one of embodiments B1-B6, wherein the encoded polypeptide is thermally stable.

D1. A vector, comprising the polynucleotide of any one of embodiments B1-B6.

D2. The vector of embodiment D1 that is an expression vector.

D3. The vector of embodiment D1 or embodiment D2, comprising the sequence of nucleotides set forth in SEQ ID NO:4.

E1. A composition, comprising one or more polypeptides of embodiments A1-A13.

E2. The composition of embodiment E1, further comprising a diluent, adjuvant, excipient or carrier.

E3. The composition of embodiment E2 comprising an adjuvant, wherein one or more polypeptides comprise a consecutive sequence of amino acids that is more than 95% identical to a corresponding consecutive sequence of amino acids set forth in SEQ ID NO: 1 or SEQ ID NO:2.

E4. The composition of embodiment E3, comprising a consecutive sequence of amino acids that is 96%, 97%, 98%, or 99% or more identical, or 100% identical, to a corresponding consecutive sequence of amino acids set forth in SEQ ID NO:1 or SEQ ID NO: 2.

E5. The composition of any one of embodiments E1-E4, comprising about 0.1% to about 10% weight/volume of the one or more polypeptides.

E6. The composition of any one of embodiments E1-E5, wherein the amount of the one or more polypeptides is effective to prevent, reduce or eliminate hyperosmotic stress in a cell.

E7. The composition of embodiment E6, wherein the amount of the one or more polypeptides is effective to increase the viability of one or more cells subjected to hyperosmotic stress by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%, 185%, 190%, 195%, 200%, 300%, 400% or more, relative to the viability of the one or more cell under hyperosmotic stress that is/are not subjected to treatment with the composition.

E8. The composition of any one of embodiments E1-E7, comprising a single polypeptide of any one of embodiments A1-A13.

E9. The composition of embodiment E8, consisting essentially of a single polypeptide of any one of embodiments A1-A13.

E10. The composition of embodiment E8, consisting of a single polypeptide of any one of embodiments A1-A13.

E11. The composition of any one of embodiments E1-E10, wherein one or more, or all, of the one or more polypeptides of embodiments A1-A13 exhibits osmoprotective activity and the composition does not comprise any other polypeptide that exhibits osmoprotective activity.

E12. The composition of any one of embodiments E1-E11, for use in the prevention, reduction or elimination of a disease, disorder or condition associated with inflammation, or with a disease, disorder or condition of the skin, mucosal membranes or hair, or for use as an antioxidant.

E13. The composition of embodiment E12, wherein the disease, disorder or condition is selected from among inflammatory bowel disease, hypernatremia, dry eye syndrome, tissue burns, dehydration, heat stroke, diabetes mellitus, diabetes insipidus, uremia, kidney disease, cardiovascular disease and liver disease.

E14. The composition of embodiment E12 or E13, wherein the disease, disorder or condition is one that is susceptible to infection, injury or damage in or on a subject.

E15. The composition of any one of embodiments E12-E14, wherein the disease or condition is associated with a tissue, a wound, a sore, an ulcer or an infection.

E16. The composition of embodiment E15, wherein the disease or condition is associated with a tissue and the tissue is epithelial or connective tissue.

E17. The composition of embodiment E15, wherein the tissue is a membrane.

E18. The composition of embodiment E17, wherein the membrane is a cutaneous membrane, mucous membrane, serous membrane or synovial membrane.

E19. The composition of any one of embodiments E15-18, wherein the tissue injury or damage comprises a sore, wound, burn, a skin ulcer, or an ulcer in the stomach mucosa or intestinal mucosa.

E20. The composition of any one of embodiments E12-E19, wherein the disease, disorder or condition is selected from among burns, sunburns, abrasions, cuts, pimples, allergic reactions, poisoning, exposure to radiation, chemotherapy, contact of the skin with a substance, mechanical friction, dehydration, bed sores, the application of a tattoo and piercing the skin, aging of the skin and/or dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, i aging of the skin, dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders and children's diseases affecting the skin (such as varicella, rubella, measles), wrinkles in the skin, rough skin, loss of elasticity in the skin, intense, large and/or numerous age spots, hyperpigmentation, epidermolysis bulosa and alopecia, including alopecia caused as a side effect of chemotherapy.

E21. The composition of any one of embodiments E1-E21, wherein the amount of the one or more polypeptides in the composition is effective to increase the viability of one or more cells subjected to hyperosmotic stress by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%, 185%, 190%, 195%, 200%, 300%, 400% or more, relative to the viability of the one or more cell under hyperosmotic stress that is/are not subjected to treatment with the composition.

E22. The composition of any one of embodiments E1-E21, wherein the composition further comprises an anti-inflammatory agent.

E23. The composition of any one of embodiments E1-E11, wherein the composition further comprises a cosmetically or pharmaceutically effective amount of at least one adjuvant selected from the group consisting of heat shock proteins other than the polypeptides of embodiments A1-A13, heat shock protein synthesis stimulating agents, acetylcholine-receptor aggregation inhibitors, muscle contraction inhibiting agents, anticholinergic agents, elastase inhibiting agents, matrix metalloproteinase inhibiting agents, melanin synthesis stimulating or inhibiting agents, whitening or depigmenting agents, propigmenting agents, self-tanning agents, anti-aging agents, NO-synthase inhibiting agents, 5a-reductase inhibiting agents, lysyl- and/or prolyl hydroxylase inhibiting agents, antioxidants, free radical scavengers and/or agents against atmospheric pollution, reactive carbonyl specie scavengers, anti-glycation agents, antihistamine agents, antiviral agents, antiparasitic agents, emulsifiers, emollients, organic solvents, liquid propellants, skin conditioners, humectants, substances that retain moisture, alpha hydroxyacids, beta hydroxyacids, moisturizers, epidermal hydrolytic enzymes, vitamins, amino acids, proteins, pigments or colorants, dyes, gelling polymers, thickeners, surfactants, softening agents, anti-wrinkle agents, agents able to reduce or treat bags under the eyes, exfoliating agents, antimicrobial agents, antifungal agents, fungistatic agents, bactericidal agents, bacteriostatic agents, agents stimulating the synthesis of dermal or epidermal macromolecules and/or capable of inhibiting or preventing their degradation, collagen synthesis-stimulating agents, elastin synthesis-stimulating agents, decorin synthesis-stimulating agents, laminin synthesis-stimulating agents, defensin synthesis-stimulating agents, aquaporin synthesis-stimulating agents, hyaluronic acid synthesis-stimulating agents, fibronectin synthesis-stimulating agents, sirtuin synthesis-stimulating agents, agents stimulating the synthesis of lipids and components of the stratum corneum, ceramides, fatty acids, agents that inhibit collagen degradation, agents that inhibit elastin degradation, agents that inhibit serine proteases such as cathepsin G, agents stimulating fibroblast proliferation, agents stimulating keratinocyte proliferation, agents stimulating adipocyte proliferation, agents stimulating melanocyte proliferation, agents stimulating keratinocyte differentiation, agents stimulating adipocyte differentiation, agents that inhibit acetylcholinesterase, skin relaxant agents, glycosaminoglycan synthesis-stimulating agents, antihyperkeratosis agents, comedolytic agents, antipsoriasis agents, DNA repair agents, DNA protecting agents, stabilizers, anti-itching agents, agents for the treatment and/or care of sensitive skin, firming agents, anti-stretch mark agents, binding agents, agents regulating sebum production, lipolytic agents or agents stimulating lipolysis, anti-cellulite agents, antiperspirant agents, agents stimulating healing, coadjuvant healing agents, agents stimulating re-epithelialization, coadjuvant re-epithelialization agents, cytokine growth factors, calming agents, anti-inflammatory and/or analgesic agents, anesthetic agents, agents acting on capillary circulation and/or microcirculation, agents stimulating angiogenesis, agents that inhibit vascular permeability, venotonic agents, agents acting on cell metabolism, agents to improve dermal-epidermal junction, agents inducing hair growth, hair growth inhibiting or retardant agents, hair loss retardant agents, preservatives, perfumes, chelating agents, vegetable extracts, essential oils, marine extracts, agents obtained from a bio-fermentation process, mineral salts, cell extracts and sunscreens, organic or mineral photoprotective agents active against ultraviolet A and/or B rays or mixtures thereof.

E24. The composition of embodiment E23, wherein the adjuvant is synthetic in origin or is a vegetable extract or comes from a biotechnological process or from a combination of a synthetic process and a biotechnological process.

E25. The composition of any one of embodiments E23 or E24, wherein the adjuvant is selected from the group consisting of anti-wrinkle and/or anti-aging agents.

E26. The composition of embodiment E25, wherein the anti-wrinkle and/or anti-aging agents are selected from the group consisting of Acetyl Hexapeptide-8, Acetyl Heptapeptide-4, Acetyl Octapeptide-3, Pehtapeptide-18, Acetyl Hexapeptide-25, Diaminopropionoyl Tripeptide-33, Tripeptide-10 Citrulline, Acetyl Tetrapeptide-5, Acetyl Tripeptide-30 Citrulline, Acetyl Tetrapeptide-30, Dimethylmethoxy Chromanol, Dimethylmethoxy Chromanyl Palmitate, Pseudoalteromonas Ferment Extract, antagonists of the Ca²⁺ channel, retinol and its derivatives, idebenone and its derivatives, Coenzyme Q10 and its derivatives, boswellic acid and its derivatives, GHK and its derivatives and/or salts, carnosine and its derivatives, DNA repair enzymes, agonists of chloride channels, the mixture of Hydrolyzed Wheat Protein, Hydrolyzed Soy Protein and Tripeptide-1, the mixture of Lisine HCl, Lecitine and Tripeptide-10 Citrulline and the mixture of Pseudoalteromonas Ferment Extract, Hydrolyzed Wheat Protein, Hydrolyzed Soy Protein, Tripeptide-10 Citrulline and Tripeptide-1.

E27. The composition of embodiment E23 or E24, wherein the adjuvant is selected from the group consisting of agents stimulating healing and re-epithelialization and coadjuvant healing and/or re-epithelialization agents.

E28. The composition of embodiment E27, wherein the agent stimulating healing and/or re-epithelialization or coadjuvant healing and/or re-epithelialization agent is selected from the group consisting of Pseudoalteromonas Ferment Extract and Tripeptide-10 Citrulline.

E29. The composition of embodiment E23 or E24, wherein the adjuvant is selected from the group consisting of hair loss retardant agents or agents inducing hair growth.

E30. The composition of embodiment E23 or E24, wherein the adjuvant is selected from the group consisting of sunscreens.

E31. The composition of any one of embodiments E1-E30, wherein the composition is in the form of a liquid, solid, semi-solid or mixture of a liquid, solid and/or semi-solid.

E32. The composition of any one of embodiments E1-E31, wherein the composition is in the form of a solution, dispersion, suspension, emulsion, or colloid.

E33. The composition of any one of embodiments E1-E32, wherein the composition is in the form of a pencil, cream, lotion, emulsion, oil, butter, paste, balm, stick, foam, gel, serum, ointment, mousse, powder, semi-solid formulation, patch, spray or aerosol.

E34. The composition of any one of embodiments E1-E33, wherein the composition is formulated for topical, oral, transdermal or parenteral administration to a subject.

E35. The composition of embodiment E34, wherein the subject is an animal.

E36. The composition of embodiment E34 or E35, wherein the subject is a human.

F1. A method of treating osmotic stress in a subject, or in one or more cells of a subject, comprising, administering to the subject, or to one or more cells of a subject, the polypeptide of any one of embodiments A1-A13, or the composition of embodiments E1-E36 in an amount that is sufficient to treat osmotic stress in the subject, or in one or more cells of the subject.

F2. The method of embodiment F1, wherein the subject has an inflammatory disorder or condition.

F3. The method of embodiment F2, wherein the disorder or condition is selected from among inflammatory bowel disease, hypernatremia, dry eye syndrome, tissue burns, dehydration, heat stroke, diabetes mellitus, diabetes insipidus, uremia, kidney disease, cardiovascular disease and liver disease.

F4. The method of any one of embodiments F1-F3, wherein the subject has a disorder or condition that is associated with the skin, mucous membranes or hair.

F5. The method of embodiment F4, wherein the disorder or condition associated with the skin, mucous membranes or hair is selected from among aging of the skin, dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders and children's diseases affecting the skin (such as varicella, rubella, measles), wrinkles in the skin, rough skin, loss of elasticity in the skin, intense, large and/or numerous age spots, hyperpigmentation, epidermolysis bulosa and alopecia, including alopecia caused as a side effect of chemotherapy.

F6. The method of any one of embodiments F1-F5, wherein the subject has a disease, disorder or condition that is susceptible to infection, injury or damage in or on a subject.

F7. The method of embodiment F6, wherein the disease or condition is associated with a tissue, a wound, a sore, an ulcer or an infection.

F8. The method of embodiment F7, wherein the disease or condition is associated with a tissue and the tissue is epithelial or connective tissue.

F9. The method of embodiment F8, wherein the tissue is a membrane.

F10. The method of embodiment F9, wherein the membrane is a cutaneous membrane, mucous membrane, serous membrane or synovial membrane.

F11. The method of any one of embodiments F8-F10, wherein the tissue injury or damage comprises a sore, wound, burn, a skin ulcer, or an ulcer in the stomach mucosa or intestinal mucosa.

F12. The method of any one of embodiments F6-F11, wherein the disease, disorder or condition is selected from among burns, sunburns, abrasions, cuts, pimples, allergic reactions, poisoning, exposure to radiation, chemotherapy, contact of the skin with a substance, mechanical friction, dehydration, bed sores, the application of a tattoo and piercing the skin, aging of the skin and/or dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, i aging of the skin, dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders and children's diseases affecting the skin (such as varicella, rubella, measles), wrinkles in the skin, rough skin, loss of elasticity in the skin, intense, large and/or numerous age spots, hyperpigmentation, epidermolysis bulosa and alopecia, including alopecia caused as a side effect of chemotherapy.

F13. The method of any one of embodiments F1-F12, wherein one or more polypeptides comprise a consecutive sequence of amino acids that is more than 95% identical to a corresponding consecutive sequence of amino acids set forth in SEQ ID NO:1 or SEQ ID NO: 2.

F14. The method of embodiment F13, wherein one or more polypeptides comprise a consecutive sequence of amino acids that is 96%, 97%, 98%, or 99% or more identical, or 100% identical, to a corresponding consecutive sequence of amino acids set forth in SEQ ID NO:1 or SEQ ID NO:2.

F15. The method of any one of embodiments F1-F14, comprising about 0.1% to about 10% weight/volume of the one or more polypeptides.

F16. The method of any one of embodiments F1-F15, wherein the amount of the one or more polypeptides of embodiments A1-A13 or the amount of the one or more polypeptides in the composition of embodiments E1-E36 is effective to prevent, reduce or eliminate hyperosmotic stress in a cell.

F17. The method of embodiment F16, wherein the amount of the one or more polypeptides is effective to increase the viability of one or more cells subjected to hyperosmotic stress by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%, 185%, 190%, 195%, 200%, 300%, 400% or more, relative to the viability of the one or more cell under hyperosmotic stress that is/are not subjected to treatment with the composition.

F18. The method of any one of embodiments F1-F17, comprising administering a single polypeptide, or a composition comprising a single polypeptide.

F19. The method of any one of embodiments F1-F18, wherein one or more, or all, of the one or more polypeptides that are administered exhibit osmoprotective activity.

F20. The method of any one of embodiments F1-F19, comprising administering the composition of any one of embodiments E1-E36.

F21. The method of embodiment F20, wherein the composition comprises a diluent, adjuvant, excipient or carrier.

F22. The method of embodiment F20 or F21, wherein the composition further comprises an anti-inflammatory agent.

F23. The method of embodiment F21 or F22, wherein the composition further comprises a cosmetically or pharmaceutically effective amount of at least one adjuvant selected from the group consisting of heat shock proteins other than the polypeptides of embodiments A1-A13, heat shock protein synthesis stimulating agents, acetylcholine-receptor aggregation inhibitors, muscle contraction inhibiting agents, anticholinergic agents, elastase inhibiting agents, matrix metalloproteinase inhibiting agents, melanin synthesis stimulating or inhibiting agents, whitening or depigmenting agents, propigmenting agents, self-tanning agents, anti-aging agents, NO-synthase inhibiting agents, 5a-reductase inhibiting agents, lysyl- and/or prolyl hydroxylase inhibiting agents, antioxidants, free radical scavengers and/or agents against atmospheric pollution, reactive carbonyl specie scavengers, anti-glycation agents, antihistamine agents, antiviral agents, antiparasitic agents, emulsifiers, emollients, organic solvents, liquid propellants, skin conditioners, humectants, substances that retain moisture, alpha hydroxyacids, beta hydroxyacids, moisturizers, epidermal hydrolytic enzymes, vitamins, amino acids, proteins, pigments or colorants, dyes, gelling polymers, thickeners, surfactants, softening agents, anti-wrinkle agents, agents able to reduce or treat bags under the eyes, exfoliating agents, antimicrobial agents, antifungal agents, fungistatic agents, bactericidal agents, bacteriostatic agents, agents stimulating the synthesis of dermal or epidermal macromolecules and/or capable of inhibiting or preventing their degradation, collagen synthesis-stimulating agents, elastin synthesis-stimulating agents, decorin synthesis-stimulating agents, laminin synthesis-stimulating agents, defensin synthesis-stimulating agents, aquaporin synthesis-stimulating agents, hyaluronic acid synthesis-stimulating agents, fibronectin synthesis-stimulating agents, sirtuin synthesis-stimulating agents, agents stimulating the synthesis of lipids and components of the stratum corneum, ceramides, fatty acids, agents that inhibit collagen degradation, agents that inhibit elastin degradation, agents that inhibit serine proteases such as cathepsin G, agents stimulating fibroblast proliferation, agents stimulating keratinocyte proliferation, agents stimulating adipocyte proliferation, agents stimulating melanocyte proliferation, agents stimulating keratinocyte differentiation, agents stimulating adipocyte differentiation, agents that inhibit acetylcholinesterase, skin relaxant agents, glycosaminoglycan synthesis-stimulating agents, antihyperkeratosis agents, comedolytic agents, antipsoriasis agents, DNA repair agents, DNA protecting agents, stabilizers, anti-itching agents, agents for the treatment and/or care of sensitive skin, firming agents, anti-stretch mark agents, binding agents, agents regulating sebum production, lipolytic agents or agents stimulating lipolysis, anti-cellulite agents, antiperspirant agents, agents stimulating healing, coadjuvant healing agents, agents stimulating re-epithelialization, coadjuvant re-epithelialization agents, cytokine growth factors, calming agents, anti-inflammatory and/or analgesic agents, anesthetic agents, agents acting on capillary circulation and/or microcirculation, agents stimulating angiogenesis, agents that inhibit vascular permeability, venotonic agents, agents acting on cell metabolism, agents to improve dermal-epidermal junction, agents inducing hair growth, hair growth inhibiting or retardant agents, hair loss retardant agents, preservatives, perfumes, chelating agents, vegetable extracts, essential oils, marine extracts, agents obtained from a bio-fermentation process, mineral salts, cell extracts and sunscreens, organic or mineral photoprotective agents active against ultraviolet A and/or B rays or mixtures thereof.

F24. The method of embodiment F23, wherein the adjuvant is synthetic in origin or is a vegetable extract or comes from a biotechnological process or from a combination of a synthetic process and a biotechnological process.

F25. The method of any one of embodiments F23 or F24, wherein the adjuvant is selected from the group consisting of anti-wrinkle and/or anti-aging agents.

F26. The method of embodiment F25, wherein the anti-wrinkle and/or anti-aging agents are selected from the group consisting of Acetyl Hexapeptide-8, Acetyl Heptapeptide-4, Acetyl Octapeptide-3, Pehtapeptide-18, Acetyl Hexapeptide-25, Diaminopropionoyl Tripeptide-33, Tripeptide-10 Citrulline, Acetyl Tetrapeptide-5, Acetyl Tripeptide-30 Citrulline, Acetyl Tetrapeptide-30, Dimethylmethoxy Chromanol, Dimethylmethoxy Chromanyl Palmitate, Pseudoalteromonas Ferment Extract, antagonists of the Ca²⁺ channel, retinol and its derivatives, idebenone and its derivatives, Coenzyme Q10 and its derivatives, boswellic acid and its derivatives, GHK and its derivatives and/or salts, carnosine and its derivatives, DNA repair enzymes, agonists of chloride channels, the mixture of Hydrolyzed Wheat Protein, Hydrolyzed Soy Protein and Tripeptide-1, the mixture of Lisine HCl, Lecitine and Tripeptide-10 Citrulline and the mixture of Pseudoalteromonas Ferment Extract, Hydrolyzed Wheat Protein, Hydrolyzed Soy Protein, Tripeptide-10 Citrulline and Tripeptide-1.

F27. The method of embodiment F23 or F24, wherein the adjuvant is selected from the group consisting of agents stimulating healing and/re-epithelialization and coadjuvant healing and/or re-epithelialization agents.

F28. The method of embodiment F27, wherein the agent stimulating healing and/or re-epithelialization or coadjuvant healing and/or re-epithelialization agent is selected from the group consisting of Pseudoalteromonas Ferment Extract and Tripeptide-10 Citrulline.

F29. The method of embodiment F23 or F24, wherein the adjuvant is selected from the group consisting of hair loss retardant agents or agents inducing hair growth.

F30. The method of embodiment F23 or F24, wherein the adjuvant is selected from the group consisting of sunscreens.

F31. The method of any one of embodiments F1-F30, wherein the administration is topical, oral, transdermal or parenteral.

F32. The method of any one of embodiments F1-F31, wherein the subject is an animal.

F33. The method of any one of embodiments F1-F32, wherein the subject is a human.

G1. A method of treating an inflammatory disease or condition in a subject, or a disorder or condition that is associated with the skin, mucous membranes or hair of a subject, comprising, administering to the subject, the polypeptide of any one of embodiments A1-A13, or the composition of embodiments E1-E36 in an amount that is sufficient to ameliorate or treating an inflammatory disease or condition in the subject, or a disorder or condition that is associated with the skin, mucous membranes or hair of the subject.

G2. The method of embodiment G1, wherein the subject has an inflammatory disorder or condition.

G3. The method of embodiment G2, wherein the disorder or condition is selected from among inflammatory bowel disease, hypernatremia, dry eye syndrome, tissue burns, dehydration, heat stroke, diabetes mellitus, diabetes insipidus, uremia, kidney disease, cardiovascular disease and liver disease.

G4. The method of any one of embodiments G1-G3, wherein the subject has a disorder or condition that is associated with the skin, mucous membranes or hair.

G5. The method of embodiment G4, wherein the disorder or condition associated with the skin, mucous membranes or hair is selected from among aging of the skin, dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders and children's diseases affecting the skin (such as varicella, rubella, measles), wrinkles in the skin, rough skin, loss of elasticity in the skin, intense, large and/or numerous age spots, hyperpigmentation, epidermolysis bulosa and alopecia, including alopecia caused as a side effect of chemotherapy.

G6. The method of any one of embodiments G1-G5, wherein the subject has a disease, disorder or condition that is susceptible to infection, injury or damage in or on a subject.

G7. The method of embodiment G6, wherein the disease or condition is associated with a tissue, a wound, a sore, an ulcer or an infection.

G8. The method of embodiment G7, wherein the disease or condition is associated with a tissue and the tissue is epithelial or connective tissue.

G9. The method of embodiment G8, wherein the tissue is a membrane.

G10. The method of embodiment G9, wherein the membrane is a cutaneous membrane, mucous membrane, serous membrane or synovial membrane.

G11. The method of any one of embodiments G8-G10, wherein the tissue injury or damage comprises a sore, wound, burn, a skin ulcer, or an ulcer in the stomach mucosa or intestinal mucosa.

G12. The method of any one of embodiments G6-G11, wherein the disease, disorder or condition is selected from among burns, sunburns, abrasions, cuts, pimples, allergic reactions, poisoning, exposure to radiation, chemotherapy, contact of the skin with a substance, mechanical friction, dehydration, bed sores, the application of a tattoo and piercing the skin, aging of the skin and/or dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, i aging of the skin, dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders and children's diseases affecting the skin (such as varicella, rubella, measles), wrinkles in the skin, rough skin, loss of elasticity in the skin, intense, large and/or numerous age spots, hyperpigmentation, epidermolysis bulosa and alopecia, including alopecia caused as a side effect of chemotherapy.

G13. The method of any one of embodiments G1-G12, wherein one or more polypeptides comprise a consecutive sequence of amino acids that is more than 95% identical to a corresponding consecutive sequence of amino acids set forth in SEQ ID NO:1 or SEQ ID NO: 2.

G14. The method of embodiment G13, wherein one or more polypeptides comprise a consecutive sequence of amino acids that is 96%, 97%, 98%, or 99% or more identical, or 100% identical, to a corresponding consecutive sequence of amino acids set forth in SEQ ID NO:1 or SEQ ID NO:2.

G15. The method of any one of embodiments G1-G14, comprising about 0.1% to about 10% weight/volume of the one or more polypeptides.

G16. The method of any one of embodiments G1-G15, wherein the amount of the one or more polypeptides of embodiments A1-A13 or the amount of the one or more polypeptides in the composition of embodiments E1-E36 is effective to prevent, reduce or eliminate hyperosmotic stress in a cell.

G17. The method of embodiment G16, wherein the amount of the one or more polypeptides is effective to increase the viability of one or more cells subjected to hyperosmotic stress by at least about 10%, 15%, 20%, 25%, 30%, 35%, 40%, 45%, 50%, 55%, 60%, 65%, 70%, 75%, 80%, 85%, 90%, 95%, 100%, 125%, 150%, 155%, 160%, 165%, 170%, 175%, 180%, 185%, 190%, 195%, 200%, 300%, 400% or more, relative to the viability of the one or more cell under hyperosmotic stress that is/are not subjected to treatment with the composition.

G18. The method of any one of embodiments G1-G17, comprising administering a single polypeptide, or a composition comprising a single polypeptide.

G19. The method of any one of embodiments G1-G18, wherein one or more, or all, of the one or more polypeptides that are administered exhibit osmoprotective activity.

G20. The method of any one of embodiments G1-G19, comprising administering the composition of any one of embodiments E1-E36.

G21. The method of embodiment G20, wherein the composition comprises a diluent, adjuvant, excipient or carrier.

G22. The method of embodiment G20 or G21, wherein the composition further comprises an anti-inflammatory agent.

G23. The method of embodiment G21 or G22, wherein the composition further comprises a cosmetically or pharmaceutically effective amount of at least one adjuvant selected from the group consisting of heat shock proteins other than the polypeptides of embodiments A1-A13, heat shock protein synthesis stimulating agents, acetylcholine-receptor aggregation inhibitors, muscle contraction inhibiting agents, anticholinergic agents, elastase inhibiting agents, matrix metalloproteinase inhibiting agents, melanin synthesis stimulating or inhibiting agents, whitening or depigmenting agents, propigmenting agents, self-tanning agents, anti-aging agents, NO-synthase inhibiting agents, 5a-reductase inhibiting agents, lysyl- and/or prolyl hydroxylase inhibiting agents, antioxidants, free radical scavengers and/or agents against atmospheric pollution, reactive carbonyl specie scavengers, anti-glycation agents, antihistamine agents, antiviral agents, antiparasitic agents, emulsifiers, emollients, organic solvents, liquid propellants, skin conditioners, humectants, substances that retain moisture, alpha hydroxyacids, beta hydroxyacids, moisturizers, epidermal hydrolytic enzymes, vitamins, amino acids, proteins, pigments or colorants, dyes, gelling polymers, thickeners, surfactants, softening agents, anti-wrinkle agents, agents able to reduce or treat bags under the eyes, exfoliating agents, antimicrobial agents, antifungal agents, fungistatic agents, bactericidal agents, bacteriostatic agents, agents stimulating the synthesis of dermal or epidermal macromolecules and/or capable of inhibiting or preventing their degradation, collagen synthesis-stimulating agents, elastin synthesis-stimulating agents, decorin synthesis-stimulating agents, laminin synthesis-stimulating agents, defensin synthesis-stimulating agents, aquaporin synthesis-stimulating agents, hyaluronic acid synthesis-stimulating agents, fibronectin synthesis-stimulating agents, sirtuin synthesis-stimulating agents, agents stimulating the synthesis of lipids and components of the stratum corneum, ceramides, fatty acids, agents that inhibit collagen degradation, agents that inhibit elastin degradation, agents that inhibit serine proteases such as cathepsin G, agents stimulating fibroblast proliferation, agents stimulating keratinocyte proliferation, agents stimulating adipocyte proliferation, agents stimulating melanocyte proliferation, agents stimulating keratinocyte differentiation, agents stimulating adipocyte differentiation, agents that inhibit acetylcholinesterase, skin relaxant agents, glycosaminoglycan synthesis-stimulating agents, antihyperkeratosis agents, comedolytic agents, antipsoriasis agents, DNA repair agents, DNA protecting agents, stabilizers, anti-itching agents, agents for the treatment and/or care of sensitive skin, firming agents, anti-stretch mark agents, binding agents, agents regulating sebum production, lipolytic agents or agents stimulating lipolysis, anti-cellulite agents, antiperspirant agents, agents stimulating healing, coadjuvant healing agents, agents stimulating re-epithelialization, coadjuvant re-epithelialization agents, cytokine growth factors, calming agents, anti-inflammatory and/or analgesic agents, anesthetic agents, agents acting on capillary circulation and/or microcirculation, agents stimulating angiogenesis, agents that inhibit vascular permeability, venotonic agents, agents acting on cell metabolism, agents to improve dermal-epidermal junction, agents inducing hair growth, hair growth inhibiting or retardant agents, hair loss retardant agents, preservatives, perfumes, chelating agents, vegetable extracts, essential oils, marine extracts, agents obtained from a bio-fermentation process, mineral salts, cell extracts and sunscreens, organic or mineral photoprotective agents active against ultraviolet A and/or B rays or mixtures thereof.

G24. The method of embodiment G23, wherein the adjuvant is synthetic in origin or is a vegetable extract or comes from a biotechnological process or from a combination of a synthetic process and a biotechnological process.

G25. The method of any one of embodiments G23 or G24, wherein the adjuvant is selected from the group consisting of anti-wrinkle and/or anti-aging agents.

G26. The method of embodiment G25, wherein the anti-wrinkle and/or anti-aging agents are selected from the group consisting of Acetyl Hexapeptide-8, Acetyl Heptapeptide-4, Acetyl Octapeptide-3, Pehtapeptide-18, Acetyl Hexapeptide-25, Diaminopropionoyl Tripeptide-33, Tripeptide-10 Citrulline, Acetyl Tetrapeptide-5, Acetyl Tripeptide-30 Citrulline, Acetyl Tetrapeptide-30, Dimethylmethoxy Chromanol, Dimethylmethoxy Chromanyl Palmitate, Pseudoalteromonas Ferment Extract, antagonists of the Ca²⁺ channel, retinol and its derivatives, idebenone and its derivatives, Coenzyme Q10 and its derivatives, boswellic acid and its derivatives, GHK and its derivatives and/or salts, carnosine and its derivatives, DNA repair enzymes, agonists of chloride channels, the mixture of Hydrolyzed Wheat Protein, Hydrolyzed Soy Protein and Tripeptide-1, the mixture of Lisine HCl, Lecitine and Tripeptide-10 Citrulline and the mixture of Pseudoalteromonas Ferment Extract, Hydrolyzed Wheat Protein, Hydrolyzed Soy Protein, Tripeptide-10 Citrulline and Tripeptide-1.

G27. The method of embodiment G23 or G24, wherein the adjuvant is selected from the group consisting of agents stimulating healing and/re-epithelialization and coadjuvant healing and/or re-epithelialization agents.

G28. The method of embodiment G27, wherein the agent stimulating healing and/or re-epithelialization or coadjuvant healing and/or re-epithelialization agent is selected from the group consisting of Pseudoalteromonas Ferment Extract and Tripeptide-10 Citrulline.

G29. The method of embodiment G23 or G24, wherein the adjuvant is selected from the group consisting of hair loss retardant agents or agents inducing hair growth.

G30. The method of embodiment G23 or G24, wherein the adjuvant is selected from the group consisting of sunscreens.

G31. The method of any one of embodiments G1-G30, wherein the administration is topical, oral, transdermal or parenteral.

G32. The method of any one of embodiments G1-G31, wherein the subject is an animal.

G33. The method of any one of embodiments G1-G32, wherein the subject is a human.

H1. A device, comprising the polypeptide of any one of embodiments A1-A13 or the composition of any one of embodiments E1-E36.

H2. The device of embodiment H1 that is a wound dressing, a topical patch, syringe, an inhaler, a dosage cup, a dropper, a pump, a spray bottle, an aerosol container or an applicator for administering the polypeptide or composition.

H3. The device of embodiment H2 that is a pump.

H4. The device of embodiment H2 that is a spray bottle or aerosol container.

J1. A kit, comprising the polypeptide of any one of embodiments A1-A13 or the composition of any one of embodiments E1-E36 and a device for administration of the composition.

J2. The kit of embodiment J1, wherein the polypeptide or composition is contained in the device for administration.

J3. The kit of embodiment J1, wherein the polypeptide or composition is present as a separate component that is distinct from the device.

J4. The kit of any one of embodiments J1-J3, wherein the device is a dressing, a topical patch, a pump, a spray bottle, an aerosol container, a syringe, an inhaler, a dosage cup, a dropper, or an applicator.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology has been described with reference to specific implementations. The terms and expressions that have been utilized herein to describe the technology are descriptive and not necessarily limiting. The terms and expressions that have been employed are used as terms of description and not of limitation and use of such terms and expressions do not exclude any equivalents of the features shown and described or portions thereof, and various modifications are possible within the scope of the technology claimed. Thus, it should be understood that although the present technology has been specifically disclosed by representative embodiments and optional features, modification and variation of the concepts herein disclosed may be resorted to by those skilled in the art, and such modifications and variations are considered within the scope of this technology.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%: e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

We claim:

1. An isolated, recombinant or synthetically produced polypeptide comprising a consecutive sequence of amino acids that is more than 95% identical to a corresponding consecutive sequence of amino acids set forth in SEQ ID NO:1 or SEQ ID NO:2.

2. The polypeptide of claim 1, comprising a consecutive sequence of amino acids that is 96%, 97%, 98%, or 99% or more identical, or 100% identical, to a corresponding consecutive sequence of amino acids set forth in SEQ ID NO: 1.

3. The polypeptide of claim 1, comprising a consecutive sequence of amino acids that is 96%, 97%, 98%, or 99% or more identical, or 100% identical, to a corresponding consecutive sequence of amino acids set forth in SEQ ID NO: 2.

4. The polypeptide of any one of claims 1-3, for use in the prevention, reduction or elimination of a disease, disorder or condition associated with inflammation, or with a disease, disorder or condition of the skin, mucosal membranes or hair, or for use as an antioxidant.

5. The polypeptide of claim 4, wherein the disease, disorder or condition is selected from among burns, sunburns, abrasions, cuts, pimples, allergic reactions, poisoning, exposure to radiation, chemotherapy, contact of the skin with a substance, mechanical friction, dehydration, bed sores, the application of a tattoo and piercing the skin, aging of the skin and/or dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, i aging of the skin, dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders and children's diseases affecting the skin (such as varicella, rubella, measles), wrinkles in the skin, rough skin, loss of elasticity in the skin, intense, large and/or numerous age spots, hyperpigmentation, epidermolysis bulosa and alopecia, including alopecia caused as a side effect of chemotherapy.

6. A composition, comprising one or more polypeptides of any one of claims 1-3 and a diluent, adjuvant, excipient, or carrier.

7. The composition of claim 6, further comprising an additional diluent, adjuvant, or excipient, or carrier.

8. The composition of claim 6, comprising about 0.1% to about 10% weight/volume of the one or more polypeptides.

9. The composition of claim 6, comprising a single species of polypeptide that is more than 95% identical to a corresponding consecutive sequence of amino acids set forth in SEQ ID NO:1 or SEQ ID NO:2.

10. The composition of claim 6, consisting essentially of a single species of polypeptide that is more than 95% identical to a corresponding consecutive sequence of amino acids set forth in SEQ ID NO:1 or SEQ ID NO:2.

11. The composition of claim 6, consisting of single species of polypeptide that is more than 95% identical to a corresponding consecutive sequence of amino acids set forth in SEQ ID NO:1 or SEQ ID NO:2, and a carrier.

12. The composition of any one of claims 6-11, for use in the prevention, reduction or elimination of a disease, disorder or condition associated with inflammation, or with a disease, disorder or condition of the skin, mucosal membranes or hair, or for use as an antioxidant.

13. The composition of claim 12, wherein the disease, disorder or condition is selected from among burns, sunburns, abrasions, cuts, pimples, allergic reactions, poisoning, exposure to radiation, chemotherapy, contact of the skin with a substance, mechanical friction, dehydration, bed sores, the application of a tattoo and piercing the skin, aging of the skin and/or dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, i aging of the skin, dehydration of the skin, acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders and children's diseases affecting the skin (such as varicella, rubella, measles), wrinkles in the skin, rough skin, loss of elasticity in the skin, intense, large and/or numerous age spots, hyperpigmentation, epidermolysis bulosa and alopecia, including alopecia caused as a side effect of chemotherapy.

14. The composition of any one of claims 6-13, wherein the adjuvant is selected from the group consisting of anti-wrinkle and/or anti-aging agents and/or sunscreens.

15. The composition of any one of claims 6-14, wherein the composition is in the form of a liquid, solid, semi-solid or mixture of a liquid and solid, solution, dispersion, suspension, emulsion, colloid, pencil, cream, lotion, oil, butter, paste, balm, stick, foam, gel, serum, ointment, mousse, powder, patch, spray or aerosol.
